SEQUENCE LISTING (42 sequences)

SEQ ID NO: 1 - protein, 327 aa, Artificial Sequence
  Chymopapain isoenzyme
MDFYTVGYSQDDLTSIERLIQLFDSWMLKHNKIYESIDEKIYRFEIFRDNLMYIDETNKKNNSYWLGLNGFADLSNDEFKKKYVGFVAEDFTGLEHFDNEDFTYKHVTNYPQSIDWRAKGAVTPVKNQGACGSCWAFSTIATVEGINKIVTGNLLELSEQELVDCDKHSYGCKGGYQTTSLQYVANNGVHTSKVYPYQAKQYKCRATDKPGPKVKITGYKRVPSNCETSFLGALANQPLSVLVEAGGKPFQLYKSGVFDGPCGTKLDHAVTAVGYGTSDGKNYIIIKNSWGPNWGEKGYMRLKRQSGNSQGTCGVYKSSYYPFKGFA

SEQ ID NO: 2 - DNA, 1106 bp, Artificial Sequence
  Chymopapain isoenzyme
cggataacaattcccctctagaaataattttgtttaactttaagaaggagatataccatggatttttacaccgtgggttattctcaagatgacttgacgtctatcgaaagacttatacaactctttgactcgtggatgttgaaacacaataagatttacgagagtattgatgaaaaaatatacagatttgagatttttagagataatctcatgtatattgatgagacaaacaaaaagaataatagctattggcttggattaaacggctttgctgatttaagcaatgatgaattcaagaaaaagtacgttggctttgttgctgaagattttacaggattagaacacttcgacaacgaagactttacttataaacatgttacgaattatcctcagagcattgattggagagcgaaaggagccgtgactcctgtaaaaaatcagggtgcttgtggtagttgttgggcattttcaactattgcaactgtcgagggaataaacaagattgtaactgggaatttactagaattatcagagcaagaattggtagattgtgacaaacatagctacgggtgtaaaggaggttaccagacaacttcacttcaatatgtagctaacaatggtgttcacacgagtaaagtgtatccgtatcaggcgaagcaatacaaatgtcgagctacagacaagccaggtccgaaggtgaaaattactgggtataaacgtgtgccgtcaaattgtgaaacatctttcttaggagcactcgcaaatcaacctttaagcgtccttgttgaagctggaggaaaaccattccagttgtataaatcgggagtttttgacggaccatgcggaaccaaattagatcatgcagtaacagccgtaggatacgggacaagtgatggaaaaaattacataatcataaagaattcatggggcccaaattggggcgaaaaaggatacatgagactgaaaagacaatctggcaacagtcaaggaacttgcggagtatataaaagttcctactatccttttaaaggatttgctaaggatcttggctttcatacatacatataactcgagcaccaccaccaccaccactgagatccggctgc

SEQ ID NO: 3 - protein, 336 aa, Artificial Sequence
  Chymopapain isoenzyme
MDFYTVGYSQDDLTSIERLIQLFDSWMLKHNKIYESIDEKIYRFEIFRDNLMYIDETNKKNNSYWLGLNGFADLSNDEFKKKYVGFVAEDFTGLEHFDNEDFTYKHVTNYPQSIDWRAKGAVTPVKNQGSCGSCWAFSTIATVEGINKIVTGNLLELSEQELVDCDKNSHGCKGGYQTTSLQYVADNGVHTSKVYPYQAKQCKCRAEDKPGPKVKISGYKRVPSNCETSFLGALANQPLSVLVEAGGKPFQLYKSGVFDGPCGTKLDHAVTAVGYGTSDGKNYIIIKNSWGSKWGEKGYMRLKRQSGNCQGTCGVYKSSYYPFKGFAKDLGFHTYI

SEQ ID NO: 4 - DNA, 1151 bp, Artificial Sequence
  Chymopapain isoenzyme
gattgtgagcggataacaattcccctctagaaataattttgtttaactttaagaaggagatataccatggatttttacaccgtgggttattctcaagatgatttgacgtctatcgaaagacttatacaactctttgactcgtggatgttgaaacacaataagatttacgagagtattgatgaaaaaatatacagatttgagatttttagagataatctcatgtatattgatgagacaaacaaaaagaataatagctattggcttggattaaacggctttgctgatttaagcaatgatgaattcaagaaaaagtacgttggctctgttgctgaagattttacaggattagaacacttcgacaacgaagactttacttataaacatgttacgaattatcctcagagcattgattggagagcgaaaggagccgtgactcctgtaaaaaatcagggttcttgtggtagttgttgggcattttcaactattgcaaccgtcgagggaataaacaagattgtaactgggaatttactagaattatcagagcaagaattggtagattgtgacaaaaatagccacgggtgtaaaggaggttaccagacaacttcacttcaatatgtagctgacaatggtgttcacacgagtaaagtgtatccgtatcaggcgaagcaatgcaaatgtcgagctgaagacaagccaggtccgaaggtgaaaatttctgggtataaacgtgtgccgtcaaattgtgaaacatctttcttaggagcactcgcaaatcaacctttaagcgtccttgttgaagctggaggaaaaccattccagttgtataaatcgggagtttttgacggaccatgcggaaccaaattagatcatgcagtaacagccgtaggatacgggacaagtgatggaaaaaattacataatcataaagaattcatggggctcaaaatggggcgaaaaaggatacatgagactgaaaagacaatctggcaactgtcagggaacttgcggagtatataaaagttcctactatccttttaaaggatttgctaaggatcttggctttcatacatacatataactcgagcaccaccaccaccaccactgagatccggctgctaacaaagcccgaaaggaagctgagtggctgctgcc

SEQ ID NO: 5 - protein, 336 aa, Artificial Sequence; identical to SEQ ID NO: 3
  Chymopapain isoenzyme

SEQ ID NO: 6 - DNA, 1151 bp, Artificial Sequence; identical to SEQ ID NO: 4
  Chymopapain isoenzyme

SEQ ID NO: 7 - protein, 352 aa, Carica papaya
MATMSSISKIIFLATCLIIHMGLSSADFYTVGYSQDDLTSIERLIQLFDSWMLKHNKIYESIDEKIYREEIFRDNLMYIDETNKKNNSYWLGLNGFADLSNDEFKKKYVGFVAEDFTGLEHFDNEDFTYKHVTNYPQSIDWRAKGAVTPVKNQGACGSCWAFSTIATVEGINKIVTGNLLELSEQELVDCDKHSYGCKGGYQTTSLQYVANNGVHTSKVYPYQAKQYKCRATDKPGPKVKITGYKRVPSNCETSFLGALANQPLSVLVEAGGKPFQLYKSGVFDGPCGTKLDHAVTAVGYGTSDGKNYIIIKNSWGPNWGEKGYMRLKRQSGNSQGTCGVYKSSYYPFKGFA

SEQ ID NO: 8 - protein, 352 aa, Carica papaya
MATMSSISKIIFLATCLIIHMGLSSADFYTVGYSQDDLTSIERLIQLFDSWMLKHNKIYESIDEKIYRFEIFRDNLMYIDETNKKNNSYWLGLNGFADLSNDEFKKKYVGFVAEDFTGLEHFDNEDFTYKHVTNYPQSIDWRAKGAVTPVKNQGACGSCWAFSTIATVEGINKIVTGNLLELSEQELVDCDKHSYGCKGGYQTTSLQYVANNGVHTSKVYPYQAKQYKCRATDKPGPKVKITGYKRVPSNCETSFLGALANQPLSFLVEAGGKPFQLYKSGVFDGPCGTKLDHAVTAVGYGTSDGKNYIIIKNSWGPNWGEKGYMRLKRQSGNSQGTCGVYKSSYYPFKGFA

SEQ ID NO: 9 - protein, 361 aa, Carica papaya
MATMSSISKIIFLATCLIIHMGLSSADFYTVGYSQDDLTSIERLIQLFDSWMLKHNKIYESIDEKIYRFEIFRDNLMYIDETNKKNNSYWLGLNGFADLSNDEFKKKYVGFVAEDFTGLEHFDNEDFTYKHVTNYPQSIDWRAKGAVTPVKNQGACGSCWAFSTIATVEGINKIVTGNLLELSEQELVDCDKHSYGCKGGYQTTSLQYVANNGVHTSKVYPCQAKQYKCRATDKPGPKVKITGYKRVPSNCETSFLGALANQPLSFLVEAGGKPFQLYKSGVFDGPCGTKLDHAVTAVGYGTSDGKNYIIIKNSWGPNWGEKGYMRLKRQSGNSQGTCGVYKSSYYPFKGFAKDLGFHTYI

SEQ ID NO: 10 - protein, 141 aa, Carica papaya
PQSIDWRAKGAVTPVKNQGACGSCWAFSTIATVEGINKIVTGNLLELSEQELVDCDRHSYGCKGGYQTTSLQYVANNGVHTSKVYPYQAKQYKCRATDKPGPKVKITGYKRVPSNCETSFLGALANQPLSVLVEAGGKPFQ

SEQ ID NO: 11 - protein, 226 aa, Carica papaya
YPQSIDWRAKGAVTPVKNQGACGSCWAFSTIATVEGINKIVTGNLLELSEQELVDCDRHSYGCKGGYQTTSLQYVANNGVHTSKVYPCQAKQYKCRATDKPGPKVKITGYKRVPSNCETSFLGALANQPLSFLVEAGGKPFQLYKSGVFDGPCGTKLDHAVTAVGYGTSDGKNYIIIKNSWGPNWGEEGYMRLKRQSGNSQGTCGVYKSSYYPFKGFAKDLGFHTY

SEQ ID NO: 12 - protein, 25 aa, Artificial Sequence
  Synthetic pre sequence
MATMSSISKIIFLATCLIIHMGLSS

SEQ ID NO: 13 - protein, 41 aa, Artificial Sequence
  Synthetic pre sequence
MLLVNQSHQGFNKEHTSKMVSAIVLYVLLAAAAHSAFAADP

SEQ ID NO: 14 - protein, 109 aa, Artificial Sequence
  Synthetic pro sequence
ADFYTVGYSQDDLTSIERLIQLFDSWMLKHNKIYESIDEKIYRFEIFRDNLMYIDETNKKNNSYWLGLNGFADLSNDEFKKKYVGFVAEDFTGLEHFDNEDFTYKHVTN

SEQ ID NO: 15 - protein, 109 aa, Artificial Sequence
  Synthetic pro sequence
MDFYTVGYSQDDLTSIERLIQLFDSWMLKHNKIYESIDEKIYRFEIFRDNLMYIDETNKKNNSYWLGLNGFADLSNDEFKKKYVGFVAEDFTGLEHFDNEDFTYKHVTN

SEQ ID NO: 16 - protein, 336 aa, Artificial Sequence
  Consensus prochymopapain
ADFYTVGYSQDDLTSIERLIQLFDSWMLKHNKIYESIDEKIYRFEIFRDNLMYIDETNKKNNSYWLGLNGFADLSNDEFKKKYVGFVAEDFTGLEHFDNEDFTYKHVTNYPQSIDWRAKGAVTPVKNQGACGSCWAFSTIATVEGINKIVTGNLLELSEQELVDCDXHSYGCKGGYQTTSLQYVANNGVHTSKVYPXQAKQYKCRATDKPGPKVKITGYKRVPSNCETSFLGALANQPLSXLVEAGGKPFQLYKSGVFDGPCGTKLDHAVTAVGYGTSDGKNYIIIKNSWGPNWGEKGYMRLKRQSGNSQGTCGVYKSSYYPFKGFAXXXXXXXXX

SEQ ID NO: 17 - protein, 227 aa, Artificial Sequence
  Consensus chymopapain sequence
YPQSIDWRAKGAVTPVKNQGACGSCWAFSTIATVEGINKIVTGNLLELSEQELVDCDXHSYGCKGGYQTTSLQYVANNGVHTSKVYPXQAKQYKCRATDKPGPKVKITGYKRVPSNCETSFLGALANQPLSXLVEAGGKPFQLYKSGVFDGPCGTKLDHAVTAVGYGTSDGKNYIIIKNSWGPNWGEKGYMRLKRQSGNSQGTCGVYKSSYYPFKGFAXXXXXXXXX

SEQ ID NO: 18 - protein, 327 aa, Carica papaya
ADFYTVGYSQDDLTSIERLIQLFDSWMLKHNKIYESIDEKIYREEIFRDNLMYIDETNKKNNSYWLGLNGFADLSNDEFKKKYVGFVAEDFTGLEHFDNEDFTYKHVTNYPQSIDWRAKGAVTPVKNQGACGSCWAFSTIATVEGINKIVTGNLLELSEQELVDCDKHSYGCKGGYQTTSLQYVANNGVHTSKVYPYQAKQYKCRATDKPGPKVKITGYKRVPSNCETSFLGALANQPLSVLVEAGGKPFQLYKSGVFDGPCGTKLDHAVTAVGYGTSDGKNYIIIKNSWGPNWGEKGYMRLKRQSGNSQGTCGVYKSSYYPFKGFA

SEQ ID NO: 19 - protein, 218 aa, Carica papaya
YPQSIDWRAKGAVTPVKNQGACGSCWAFSTIATVEGINKIVTGNLLELSEQELVDCDKHSYGCKGGYQTTSLQYVANNGVHTSKVYPYQAKQYKCRATDKPGPKVKITGYKRVPSNCETSFLGALANQPLSVLVEAGGKPFQLYKSGVFDGPCGTKLDHAVTAVGYGTSDGKNYIIIKNSWGPNWGEKGYMRLKRQSGNSQGTCGVYKSSYYPFKGFA

SEQ ID NO: 20 - protein, 327 aa, Carica papaya
ADFYTVGYSQDDLTSIERLIQLFDSWMLKHNKIYESIDEKIYRFEIFRDNLMYIDETNKKNNSYWLGLNGFADLSNDEFKKKYVGFVAEDFTGLEHFDNEDFTYKHVTNYPQSIDWRAKGAVTPVKNQGACGSCWAFSTIATVEGINKIVTGNLLELSEQELVDCDKHSYGCKGGYQTTSLQYVANNGVHTSKVYPYQAKQYKCRATDKPGPKVKITGYKRVPSNCETSFLGALANQPLSFLVEAGGKPFQLYKSGVFDGPCGTKLDHAVTAVGYGTSDGKNYIIIKNSWGPNWGEKGYMRLKRQSGNSQGTCGVYKSSYYPFKGFA

SEQ ID NO: 21 - protein, 218 aa, Carica papaya
YPQSIDWRAKGAVTPVKNQGACGSCWAFSTIATVEGINKIVTGNLLELSEQELVDCDKHSYGCKGGYQTTSLQYVANNGVHTSKVYPYQAKQYKCRATDKPGPKVKITGYKRVPSNCETSFLGALANQPLSFLVEAGGKPFQLYKSGVFDGPCGTKLDHAVTAVGYGTSDGKNYIIIKNSWGPNWGEKGYMRLKRQSGNSQGTCGVYKSSYYPFKGFA

SEQ ID NO: 22 - protein, 336 aa, Carica papaya
ADFYTVGYSQDDLTSIERLIQLFDSWMLKHNKIYESIDEKIYRFEIFRDNLMYIDETNKKNNSYWLGLNGFADLSNDEFKKKYVGFVAEDFTGLEHFDNEDFTYKHVTNYPQSIDWRAKGAVTPVKNQGACGSCWAFSTIATVEGINKIVTGNLLELSEQELVDCDKHSYGCKGGYQTTSLQYVANNGVHTSKVYPCQAKQYKCRATDKPGPKVKITGYKRVPSNCETSFLGALANQPLSFLVEAGGKPFQLYKSGVFDGPCGTKLDHAVTAVGYGTSDGKNYIIIKNSWGPNWGEKGYMRLKRQSGNSQGTCGVYKSSYYPFKGFAKDLGFHTYI

SEQ ID NO: 23 - protein, 227 aa, Carica papaya
YPQSIDWRAKGAVTPVKNQGACGSCWAFSTIATVEGINKIVTGNLLELSEQELVDCDKHSYGCKGGYQTTSLQYVANNGVHTSKVYPCQAKQYKCRATDKPGPKVKITGYKRVPSNCETSFLGALANQPLSFLVEAGGKPFQLYKSGVFDGPCGTKLDHAVTAVGYGTSDGKNYIIIKNSWGPNWGEKGYMRLKRQSGNSQGTCGVYKSSYYPFKGFAKDLGFHTYI

SEQ ID NO: 24 - protein, 227 aa, Carica papaya
YPQSIDWRAKGAVTPVKNQGACGSCWAFSTIATVEGINKIVTGNLLELSEQELVDCDKHSYGCKGGYQTTSLQYVANNGVHTSKVYPYQAKQYKCRATDKPGPKVKITGYKRVPSNCETSFLGALANQPLSVLVEAGGKPFQLYKSGVFDGPCGTKLDHAVTAVGYGTSDGKNYIIIKNSWGPNWGEKGYMRLKRQSGNSQGTCGVYKSSYYPFKGFAKDLGFHTYI

SEQ ID NO: 25 - protein, 227 aa, Carica papaya
YPQSIDWRAKGAVTPVKNQGSCGSCWAFSTIATVEGINKIVTGNLLELSEQELVDCDKNSHGCKGGYQTTSLQYVADNGVHTSKVYPYQAKQCKCRAEDKPGPKVKISGYKRVPSNCETSFLGALANQPLSVLVEAGGKPFQLYKSGVFDGPCGTKLDHAVTAVGYGTSDGKNYIIIKNSWGSKWGEKGYMRLKRQSGNCQGTCGVYKSSYYPFKGFAKDLGFHTYI

SEQ ID NO: 26 - DNA, 39 bp, Artificial Sequence
  Synthetic PCR primer
ttgttgttccatggatttttacaccgtgggttattcttc

SEQ ID NO: 27 - DNA, 40 bp, Artificial Sequence
  Synthetic PCR primer
ttgttgttctcgagagcaaaacctttaaaaggataatatg

SEQ ID NO: 28 - DNA, 43 bp, Artificial Sequence
  Synthetic PCR primer
ttgttgttctcgagtcaagcaaaacctttaaaaggataatatg

SEQ ID NO: 29 - DNA, 39 bp, Artificial Sequence; identical to SEQ ID NO: 26
  Synthetic PCR primer

SEQ ID NO: 30 - DNA, 39 bp, Artificial Sequence
  Synthetic PCR primer
ttgttgttctcgagggcgaatcctttaaaaggatagtag

SEQ ID NO: 31 - DNA, 42 bp, Artificial Sequence
  Synthetic PCR primer
ttgttgttctcgagtcaggcgaatcctttaaaaggatagtag

SEQ ID NO: 32 - DNA, 39 bp, Artificial Sequence; identical to SEQ ID NO: 26
  Synthetic PCR primer

SEQ ID NO: 33 - DNA, 39 bp, Artificial Sequence
  Synthetic PCR primer
ttgttgttctcgagtatgtatgtatgaaagccaagatcc

SEQ ID NO: 34 - DNA, 42 bp, Artificial Sequence
  Synthetic PCR primer
ttgttgttctcgagttatatgtatgtatgaaagccaagatcc

SEQ ID NO: 35 - protein, 336 aa, Artificial Sequence; identical to SEQ ID NO: 16
  Consensus prochymopapain

SEQ ID NO: 36 - protein, 336 aa, Artificial Sequence; identical to SEQ ID NO: 16
  Consensus prochymopapain

SEQ ID NO: 37 - protein, 336 aa, Artificial Sequence; identical to SEQ ID NO: 16
  Consensus prochymopapain

SEQ ID NO: 38 - protein, 336 aa, Artificial Sequence; identical to SEQ ID NO: 16
  Consensus prochymopapain

SEQ ID NO: 39 - protein, 227 aa, Artificial Sequence; identical to SEQ ID NO: 17
  Consensus chymopapain

SEQ ID NO: 40 - protein, 227 aa, Artificial Sequence; identical to SEQ ID NO: 17
  Consensus chymopapain

SEQ ID NO: 41 - protein, 227 aa, Artificial Sequence; identical to SEQ ID NO: 17
  Consensus chymopapain

SEQ ID NO: 42 - protein, 227 aa, Artificial Sequence; identical to SEQ ID NO: 17
  Consensus chymopapain

(19) United States
(12) Patent Application Publication
(10) Pub. No.: US 2008/0107705 A1
(43) Pub. Date: May 8, 2008

(54) Chymopapain Isoenzymes, Compositions, and Uses Thereof

(75) Inventor: Daniel J. McDermott, North Oaks, MN (US)

Correspondence Address: MERCHANT & GOULD PC, P.O. BOX 2903, MINNEAPOLIS, MN, 55402-0903, US

(73) Assignee: Spinal Therapies LLC, NORTH OAKS MINNESOTA (US)

(21) Appl. No.: 11/659,184

(22) PCT Filed: Jul. 29, 2005
(86) PCT No.: PCT/US05/27309

§ 371 Date: Mar. 28, 2007

Related U.S. Application Data
(60) Provisional application No. 60/603,151, filed on Aug. 4, 2004.

(51) Int. Cl. A61F 2/00; C07H 21/04; C07K 16/00; C12N 15/00; C12N 9/50; A61P 43/00; C12N 5/00; C12N 5/02; A61K 38/46

(52) U.S. Cl. 424/423; 424/94.65; 435/219; 536/23.2; 435/320.1; 435/325; 435/348; 435/255.1; 530/387.1

(57) ABSTRACT

The present invention provides novel chymopapain isoenzymes, compositions thereof, and methods for locally ad-ministering recombinant chymopapain or synthetic chymopapain to treat a disc disorder. Recombinant chymopapain or synthetic chymopapain useful in the methods of the invention can be naturally occurring chymopapain or variant chymopapain, including the novel chymopapain isoenzmyes of the invention.

This application is being filed on 29 Jul. 2005 as a PCT International Patent application in the name of Daniel J. McDermott, a U.S. citizen, applicant for the designation of all countries, and claims priority to U.S. Provisional Application Ser. No. 60/603,151, filed Aug. 4, 2004.

### BACKGROUND OF THE INVENTION

Chymopapain is a cysteine protease that is a major proteolytic component of the latex of Carica papaya. Chymopapain was first characterized and described by Jansen and Balls, 1941, J. Biol. Chem., 137:459-460 and in U.S. Pat. No. 2,313,875. Chymopapain is approved for use in chemonucleolysis, a procedure in which the enzyme is administered percutaneously via a needle into a disc having a herniated nucleus pulposus (HNP) resulting in temporary dissolution of the nucleus pulposus. Patients with a documented HNP accompanied by physical findings and symptoms, primarily sciatica, that are unresponsive to conservative care are candidates for chemonucleolysis.

Chymopapain is specific in its dissolution of glycosoaminoglycans comprising the nucleus pulposus and the breakdown products of these glycosoaminoglycans are found in the urine while the annulus fibrosus is not affected. Chemonucleolysis is a proven minimally invasive, cost effective, conservative treatment modality that can be applied prior to more invasive surgical interventions such as, for example, laminectomy, disectomy, microdiscectomy, or percutaneous discectomy (Simmons et al., 2001, Eur. Spine J, 10:192-202). At least 70% of chemonucleolysis patients will not require surgical intervention (Waddell et al. “Surgical Treatment of Lumbar Disc Prolapse and Degenerative Lumbar Disc Disease.” Neck and BackPain: The Scientific Evidence of Causes, Diagnosis and Treatment. Ed. Alf Nachemson and Egon Jonsson. Philadelphia: Lipincott Williams & Wilkins, 2000. 305-325.)

Chymopapain preparations approved for clinical use contain refined chymopapain (i.e. naturally occurring chymopapain purified from the latex of C. papaya.). Five isoforms of naturally occurring chymopapain have been described. Taylor et al., 1999, Plant Sci., 145:41-47. Refined chymopapain can contain other papaya cysteine proteases, such as caricain, papain, and glcyl endopeptidase. For example, CHYMODIACTIN® contains approximately 70 g of chymopapain per 100 g of protein, 20 g of caricain per 100 g of protein, 0.1 g of papain per 10 g of protein, and 4 g of glcyl endopeptidase per 100 g of protein. Dando et al., 1995, Spine, 20:981-985.

Less than one percent of patients receiving intradiscal injection of refined chymopapain develop symptoms of anaphylactic reaction. Dando et al., 1995, Spine, 20:981-985; Simmons et al., 2001, Eur. Spine J, 10:192-202. IgE antibodies are a major immunologic mediator of anaphylactic hypersensitivity. Patients with high serum levels of IgE antibodies against refined chymopapain are typically excluded from chemonucleolysis treatment. In these patients, antibodies to caricain and glycl endopeptidase account for approximately 75% of the total IgE antibodies against papaya cysteine proteases (e.g. chymopapain, caricain, papain, and glycl endopeptidase). Dando et al., 1995, Spine, 20:981-985.

Alternative forms of chymopapain are needed to treat patients suffering from sciatica secondary to herniated nucleus pulposus of the lumbar spine that are commonly excluded from chemonucleolysis treatment.

### SUMMARY OF THE INVENTION

The present invention provides novel recombinant or synthetic chymopapain isoenzymes, compositions thereof, and methods for locally administering recombinant chymopapain or synthetic chymopapain to treat a disc disorder.

One aspect of the present invention is methods for treating a disc disorder that include locally administering recombinant or synthetic chymopapain to a disc. Examples of disc disorders include, but are not limited to, increased intradiscal pressure, herniated nucleus pulposus, extruded nucleus pulposus, degenerative disc disease, nerve root compression or stretching, sciatica, or scoliosis. The disc can be a thoracic intervertebral disc, cervical intervertebral disc, lumbar intervertebral disc, or lumbosacral intervertebral disc. Another aspect of the invention is methods for reducing intradiscal pressure in an intervertebral spinal disc that include locally administering recombinant or synthetic chymopapain to the disc. Another aspect of the invention is methods for treating a herniated or extruded intervertebral spinal disc that include locally administering recombinant or synthetic chymopapain to the herniated or extruded disc. Another aspect of the invention is methods for decreasing spinal stiffness or increasing spinal flexibility that include locally administering recombinant or synthetic chymopapain to a disc or discs at the level of the spinal stiffness or at the abnormal curvature in scoliosis. Another aspect of the invention is methods for hydrolyzing and/or removing a nucleus pulposus from a disc that include locally administering recombinant or synthetic chymopapain to the nucleus pulposus.

In the methods of the invention, locally administering includes contacting the nucleus pulposus of the disc with recombinant chymopapain and/or synthetic chymopapain. Contacting the nucleus pulposus includes inserting a delivery device into a nucleus pulposus of the disc, confirming placement of the delivery device with an imaging device, and delivering the recombinant chymopapain and/or synthetic chymopapain in the nucleus pulposus. Examples of delivery devices include, but are not limited to, a canula, endoscope with operating channel, needle, or catheter attached to a syringe. Examples of imaging devices include radiographic imaging devices such as x-ray, ultrasound, computerized topography scanners, and magnetic resonance imagers.

Recombinant chymopapain or synthetic chymopapain useful in the methods of the invention can be prochymopapain or mature chymopapain. The recombinant or synthetic chymopapain can be naturally occurring or variant. Recombinant or synthetic chymopapain useful in the methods of the invention can have at least about 80% amino acid sequence identity with a chymopapain reference sequence. Examples of useful recombinant or synthetic chymopapain include, but are not limited to, chymopapain having an amino acid sequence of SEQ ID NO:1, 3, 5, 7, 8, 9, 10, 18, 19, 21, 21, 22, 23, 24, or 25.

Another aspect of the invention is novel chymopapain isoenzymes, polynucleotides encoding the isoenzymes, and compositions thereof. The isoenzymes of the invention can be prochymopapain or mature chymopapain, recombinant or synthetic. The isoenzymes of the invention can be naturally occurring chymopapain or variant chymopapain. The isoenzymes of the invention have at least about 80% amino acid sequence identity with a chymopapain reference sequence. In an embodiment, an isoenzyme of the invention includes an amino acid sequence of SEQ ID NO:1, 3, 5, 24, or 25. The reference sequence can be a prochymopapain sequence or a mature chymopapain sequence. The isoenzymes of the invention are useful for treating a disc disorder.

Isoenzymes of the invention can be variants of prochymopapain. In an embodiment, isoenzymes of the invention can have substitution of one or more amino acid residues corresponding to residues 1, 130, 167, 168, 170, 186, 197, 202, 207, 217, 241, 292, 293, or 309 of SEQ ID NO:16, wherein the isoenzymes have at least about 80% amino acid identity to an amino acid sequence of SEQ ID NO:16 and cysteine protease activity. Amino acid substitutions can include A1M, A130S, K167R, H168N, Y170H, N186D, Y197C Y202C, T207E, T217S, V241F, P292S, N293K, and/or S309C. In an embodiment, an isoenzyme of the invention includes an amino acid sequence of SEQ ID NO: 1, 3, or 5.

Isoenzymes of the invention can be variants of mature chymopapain. In an embodiment, isoenyzmes of the invention include substitution of one or more amino acid residues corresponding to residues 21, 58, 59, 61, 77, 88, 93, 98, 108, 132, 183, 184, or 200 of SEQ ID NO: 17 wherein the isoenzymes have at least about 80% sequence identity to an amino acid sequence of any one of SEQ ID NO:17, 39, 40, 41, or 42 and cysteine protease activity. Amino acid substitutions can include A21S, K58R, H₅₉N, Y61H, N77D, Y88C, Y93C, T98E, T108S, V132F, P183N, N184K, and/or S200C. In an embodiment, an isoenzyme of the invention includes an amino acid sequence of SEQ ID NO:25.

The present invention also provides anti-chymopapain antibodies. The antibodies can be polyclonal or monoclonal. The antibodies are useful for purifying chymopapain isoenzymes of the invention. In an embodiment, the antibodies of the invention specifically bind particular chymopapain isoenzymes and do not crossreact with other chymopapain isoenzymes or naturally occurring chymopapain.

### BRIEF DESCRIPTION OF THE DRAWINGS

FIG. 1 shows the amino acid sequence of chymopapain isoenzyme 1 (CI1; SEQ ID NO:1). The prosequence is underlined. Mature CI1 includes amino acid residues 110-337.

FIG. 2 shows a polynucleotide (SEQ ID NO:2) encoding CI1. The start and stop codons are underlined.

FIG. 3 shows the amino acid sequence of chymopapain isoenzyme 2 (CI2; SEQ ID NO:3). The prosequence is underlined. Mature CI2 (SEQ ID NO:24) includes amino acid residues 110-337.

FIG. 4 shows a polynucleotide (SEQ ID NO:4) encoding CI2. The start and stop codons are underlined.

FIG. 5 shows the amino acid sequence of chymopapain isoenzyme 3 (CI3; SEQ ID NO:5). The prosequence is underlined. Mature CI3 (SEQ ID NO:25) includes amino acid residues 110-337.

FIG. 6 shows a polynucleotide (SEQ ID NO:6) encoding Cl₂. The start and stop codons are underlined.

FIGS. 7A and 7B show an amino acid alignment of five naturally occurring chymopapain isoforms (chymopapain I (SEQ ID NO:7), chymopapain II (SEQ ID NO:8), chymopapain III (SEQ ID NO:9), chymopapain IV (SEQ ID NO:10), chymopapain V (SEQ ID NO:11) (Taylor et al., 1999, Plaint Sci., 145:41-47)) and chymopapain isoenzymes of the invention (CI1, CI2, and CI3). Signal sequences are underlined. Amino acid substitutions were determined by comparing the amino acid sequences to chymopapain I (SEQ ID NO:7). Amino acid substitutions are bolded and underlined. A single arrow indicates the start of the prosequence. A double arrow indicates the start of the mature sequence. Asterisks denote positions that have amino acid substitutions. Amino acid residues forming the catalytic triad are boxed.

FIG. 8 shows SDS-PAGE analysis of conditioned media from Sf21 cell cultures expressing a control protein or chymopapain isoenzymes of the invention. Lane 2 contains media from control-virus infected cells. The control virus expressed β-galactosidase intracellularly. Lane 3 contains media from cells infected with recombinant baculovirus expressing the prepro form of CI1. Lane 4 contains media from cells infected with recombinant baculovirus expressing the prepro form of CI2. Lane 5 contains media from cells infected with recombinant baculovirus expressing the prepro form of CI3. The pro form of CI1, CI2, and CI3 was the chymopapain form secreted to the media.

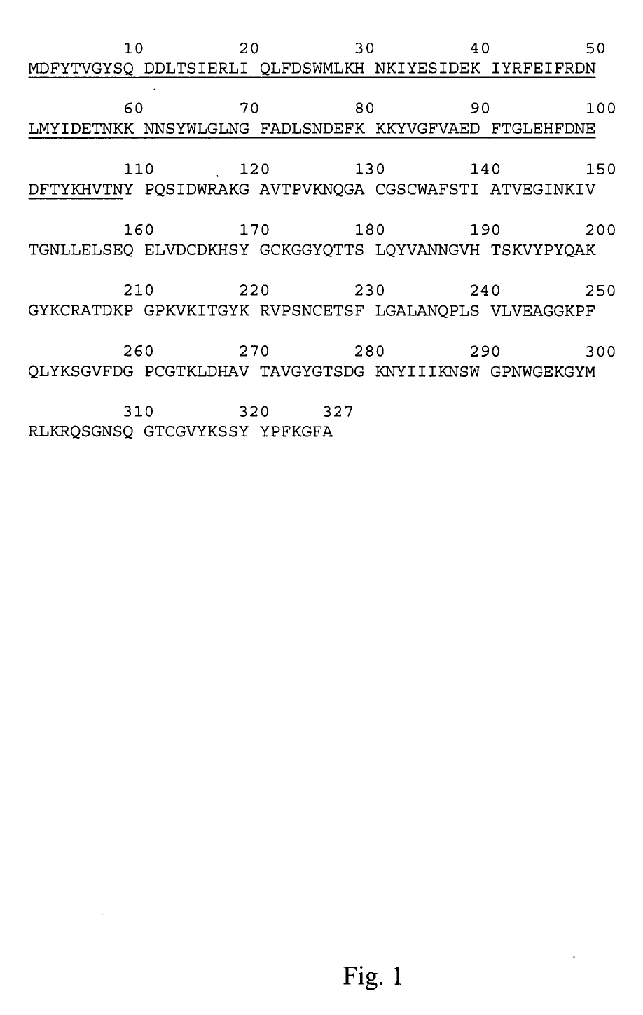

### DETAILED DESCRIPTION OF THE PREFERRED EMBODIMENTS

### I. Definitions

An “intervertebral disc” or “disc” is a cartilaginous joint between adjacent spinal vertebrae in a mammal, such as a human or dog. The disc consists of an inner, eccentrically located nucleus pulposus surrounded by a peripheral annulus fibrosus. The nucleus pulposus contains glycosoaminoglycans, proteoglycans, and hyaluronic long chains that have highly hydrophilic branching side chains. The negatively charged side chains have strong avidity for water molecules and hydrate the nucleus of the disc.

“Disc disorder” means a disorder of an intervertebral disc. A disc disorder can involve herniation or extrusion of the nucleus pulposus. Examples of a disc disorder include, but are not limited to, herniated nucleus pulposus, extruded nucleus pulposus, sciatica, scoliosis, spinal stiffness, increased intradiscal pressure, nerve root compression or stretching, chronic low back pain, herniated lumbar disc disease, or degenerative disc disease. A disc disorder can be associated with trauma to the spine.

The terms “herniated disc” and “herniated intervertebral disc” mean an intervertebral disc that has a herniated nucleus pulposus. The intervetrebral disc can be a thoracic, cervical, lumbar disc, or lumbosacral disc.

“Chemonucleolysis” refers to a procedure that includes injecting chymopapain into a nucleus pulposus resulting in dissolution of the nucleus pulposus, which can be restored in 3-6 months. Enzymatic dissolution of proteoglycans in the nucleus pulposus reduces the water-binding capacity of the disc reducing intradiscal pressure (Bradford et al., 1983, J. Bone Joint Sur., 65:1220-1231). Chemonucleolysis can include inserting a delivery device, such as for example a needle or catheter, into the nucleus pulposus of the disc, confirming placement of the needle with an imaging device, such as for example x-ray, ultrasound, computerized topography (CT scan), or magnetic resonance imaging (MRI); and injecting chymopapain or a composition containing chymopapain into the nucleus pulposus. The delivery device, such as for example a needle or catheter, can be inserted into the nucleus pulposus percutaneously or through a surgical incision.

The term “refined chymopapain” means chymopapain that has been purified from the latex of C. papaya. Refined chymopapain can be purified from the latex of C. papaya by, for example, salt fractionation, solvent fractionation, and/or chromatography methods. See, for example, U.S. Pat. Nos. 3,558,433 and 4,374,926. Refined chymopapain contains an amount of other papaya cysteine proteases, such as caricain, papain, and/or glcyl endopeptidase. It has been reported that chymopapain purified from C. papaya by chromatography methods contains approximately 70 g of chymopapain per 100 g of protein, 20 g of caricain per 100 g of proteins, 0.1 g of papain per 100 g of protein, and 4 g of glcyl endopeptidase per 100 g of protein (Dando et al., 1995, Spine, 20:981-985). The term “refined chymopapain” specifically encompasses naturally occurring prochymopapain and mature chymopapain purified from C. papaya. The term “refined chymopapain” does not encompass chymopapain that is recombinant or synthetic.

As used herein, the term “chymopapain” encompasses preprochymopapain, prochymopapain, and mature chymopapain. Unless stated otherwise, the term “chymopapain” is separate and distinct from the term “refined chymopapain.” The term “chymopapain” specifically encompasses chymopapain isoenzymes, naturally occurring chymopapain (e.g. preprochymopapain, prochymopapain, and mature chymopapain), and variants thereof. The pre region of preprochymopapain can include a secretory signal sequence. In an embodiment, the pre region includes an amino acid sequence of MATMSSISKIIFLATCLIIHMGLSS (SEQ ID NO:12). In another embodiment, the pre region includes an amino acid sequence of MLLVNQSHQGFNKEHTSKMVSAIVLYVLLAAAAHSAFAADP (SEQ ID NO:13). It has been reported that the pre region is cleaved before prochymopapain is synthesized and co-translationally translocated (Taylor et al., 1995, Protein Eng., 8:59-62). The pro region of preprochymopapain and prochymopapain can inhibit the protease activity of the mature chymopapain. In an embodiment, the pro region includes an amino acid sequence of ADFYTVGYSQDDLTSIERLIQLFDSWMLKHNKIYESIDEKIYRFEIFRDNLMY IDETNKKNNSYWLGLNGFADLSNDEFKKKYVGFVAEDFTGLEHFDNEDFTY KHVTN (SEQ ID NO:14). In another embodiment, the pro region includes an amino acid sequence of MDFYTVGYSQDDLTSERLIQLFDSWMLKBNKIYESIDEKIYRFEIFRDNLMY IDETNKKNNSYWLGLNGFADLSNDEFKKKYVGFVAEDFTGLEHFDNEDFTY KHVTN (SEQ ID NO:15). It has been reported that activation of chymopapain occurs by cleavage and removal of the pro region yielding active, mature chymopapain (Taylor et al., 1999, Plant Sci., 145(41-47). Mature chymopapain catalyses conversion of a substrate to a product via covalent catalysis, such as nucleophilic catalysis by an active site residue. An example of such catalytic conversion is hydrolysis of an amide bond of a substrate. In an embodiment, mature chymopapain has cysteine protease activity.

The term “naturally occurring chymopapain” encompasses polypeptides that have the same amino acid sequence of a chymopapain polypeptide obtained from nature. Chymopapain is found in nature in bromeliads, such as for example papaya and pineapple. Naturally occurring chymopapain amino acid sequences are disclosed, for example, in Taylor et al., 1999, Plant Sci., 145:41-47. Naturally occurring chymopapain specifically encompasses any of the naturally occurring forms of the polypeptides including naturally occurring polypeptides with a signal sequence, pro and mature forms with or without a signal sequence, truncated forms, and variant forms. Naturally occurring variants include secreted forms, alternatively spliced forms, and those naturally occurring variants that differ in sequence from a reference sequence for chymopapain. In an embodiment, the reference sequence is a prochymopapain sequence comprising an amino acid sequence of any one of SEQ ID NO:16, 35, 36, 37, or 38. In another embodiment, the reference sequence is a mature chymopapain sequence comprising an amino acid sequence of any one of SEQ ID NO 17, 39, 40, 41, or 42. Naturally occurring chymopapain can be isolated or purified from nature or prepared recombinantly or synthetically. Preferably the mature form of a naturally occurring chymopapain has cysteine protease activity.

A polypeptide that has “cysteine protease activity” catalyzes hydrolysis of an amide bond. A polypeptide that has cysteine protease activity contains a catalytic triad comprising Cys, His, and Asn. In an embodiment, the catalytic triad corresponds to Cys 25, His 159, and Asn 179 of SEQ ID NO:17. Cysteine protease activity can be detected with a substrate that has a chromogenic or fluorogenic leaving group by detecting release of the chromogenic or fluorogenic leaving group. A number of cysteine protease substrates comprising a chromogenic or fluorogenic leaving group are known in the art and commercially available, including N-α-benzoyl-DL-arginine p-nitroanilide (BAPNA) which contains a chromogenic p-nitroaniline leaving group, benzyloxycarbonyl-Phe-Arg-AMC which contains a fluorogenic 7-amino-4-methylcoumarin (AMC) leaving group, and Ac-Leu-Thr-Phe-Lys-ACC which contains a fluorogenic 7-amino-4-carbamoylmethylcoumarin (ACC) leaving group.

The term “isolated,” when used to describe the various chymopapain isoenzyme polypeptides disclosed herein, means a polypeptide that has been identified and separated and/or recovered from a component of its natural environment. Preferably, the isolated polypeptide is free of association with at least one component with which it is naturally associated. Contaminant components of its natural environment are materials that would typically interfere with diagnostic or therapeutic uses for the polypeptide and can include enzymes, and other proteinaceous or non-proteinaceous solutes, such as for example caricain, papain, and/or glcyl endopeptidase. An isolated polypeptide includes polypeptide in situ within recombinant cells, since at least one component of the chymopapain natural environment will not be present. Ordinarily, however, an isolated polypeptide will be prepared by at least one purification step.

Polynucleotide means a sequence of nucleic acids that encode a polypeptide, such as a chymopapain isoenzyme of the invention.

As used herein “recombinant” refers to a polypeptide or polynucleotide that has been isolated and/or altered by the hand of man. Typically a DNA sequence encoding a polypeptide is isolated and combined with other control sequences in a vector. The other control sequences can be those that are found in the naturally occurring gene or others. The vector provides for introduction into host cells, amplification of the polynucleotide and expression of the polynucleotide.

Peptide sequences defined herein are represented by one-letter symbols for amino acid residues as follows:

| | | | |
|---|---|---|---|
| A | alanine | L | leucine |
| R | arginine | K | lysine |
| N | asparagine | M | methionine |
| D | aspartic acid | F | phenylalanine |
| C | cysteine | P | proline |
| Q | glutamine | S | serine |
| E | glutamic acid | T | threonine |
| G | glycine | W | tryptophan |
| H | histidine | Y | tyrosine |
| I | isoleucine | V | valine |

“Percent (%) amino acid sequence identity” means the percentage of amino acid residues in a polypeptide that are identical with amino acids in a reference polypeptide, after aligning the sequence and introducing gaps, if necessary to achieve the maximum sequence identity, and not considering any conservative substitutions as part of the sequence identity. For purposes herein, the % amino acid sequence identity of a given amino acid sequence A to, with, or against a given amino acid sequence B (which can alternatively be phrased as a given amino acid sequence A that has or includes a certain % amino acid sequence identity to, with, or against a given amino acid sequence B) is calculated as follows:

100 times the fraction X/Y

where X is the number of amino acid residues scored as identical matches by the sequence alignment, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

The % amino acid sequence identity can also be determined using the sequence comparison program such as ALIGN 2 or NCBI-BLAST2 (Altschul et al., 1997, Nucleic Acids Res., 25:3389-3402). The NCBI-BLAST2 sequence comparison program can be downloaded from http://www-ncbi-nlm-nih-gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

A nucleic acid is “operably linked,” as used herein, when it is placed into a functional relationship with another nucleic acid sequence. For example, DNA for a presequence or secretory leader is operably linked to DNA encoding chymopapain if it is expressed as a preprotein that participates in the secretion of chymopapain; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, synthetic oligonucleotide adaptors or linkers are used in accordance with conventional practice.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoli/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS, and 10% dextran sulfate at 42° C., with washes at 42° C. in 0.2×SW (sodium chloride/sodium citrate) and 50% formamide at 55° C., followed by a high-stringency wash consisting of 0.1×SSC containing EDTA at 55° C.

“Moderately stringent conditions” can be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “antibody” is used in the broadest sense and specifically covers monoclonal antibodies and polyclonal antibodies.

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to conventional polyclonal antibody preparations that typically include different antibodies directed against different epitopes, each monoclonal antibody is directed against a single epitope on the antigen. The modifier “monoclonal” indicates the character of the antibody as being obtained from a substantially homogeneous population of antibodies, and is not to be construed as requiring production of the antibody by any particular method. For example, the monoclonal antibodies of the present invention can be made by the hybridoma method first described by Kohler et al., 1975, Nature, 256:495, or can be made by recombinant DNA methods (see, e.g., U.S. Pat. No. 4,816,567). The “monoclonal antibodies” can also be isolated from phage antibody libraries using the techniques described in Clackson et al., 1991, Nature, 352:624-628 and Marks et al., 1991, J. Mol. Biol., 222:581-597, for example.

An “isolated” antibody is one that has been identified and separated and/or recovered from a component of its natural environment. Contaminant components of its natural environment are materials that would interfere with diagnostic uses for the antibody, and can include enzymes, hormones, and other proteinaceous or nonproteinaceous solutes. Isolated antibody includes the antibody in situ within recombinant cells since at least one component of the antibody's natural environment will not be present. Ordinarily, however, isolated antibody will be prepared by at least one purification step.

As used herein, the term “treating” refers to therapeutic treatment. Treating includes local administration of chymopapain that alleviates, eliminates, or inhibits the progression of one or more pathological hallmarks including, but not limited to, all or some of the physical findings of and/or all or some of the symptoms of a disc disorder or a disease or disorder secondary to a disc disorder. Examples of a disease or disorder secondary to a disc disorder include, but are not limited to, a herniated disc that is secondary to increased intradiscal pressure, nerve root compression or stretching that is secondary to a herniated disc, and sciatica that is secondary to nerve root compression or stretching. Those in need of treatment include patients who have sciatica, scoliosis, spinal stiffness, increased intradiscal pressure, nerve root compression or stretching, a herniated nucleus pulposus, or degenerative disc disease. A herniated nucleus pulposus can be associated with trauma to the spine, sciatica, herniated lumbar disc disease, nerve root compression or stretching, or chronic low back pain.

The term “therapeutically effective amount” refers to an amount of a chymopapain or a composition thereof effective to treat a disc disorder, for example sciatica, scoliosis, spinal stiffness, in a mammal, such as a human or dog. A herniated nucleus pulposus can be associated with trauma to the spine, sciatica, herniated lumbar disc disease, nerve root compression or stretching, or chronic low back pain. With respect to a herniated disc, therapeutically effective amount is a dose that provides some therapeutic benefit on administration, including, in the context of the invention, reducing intradiscal pressure and/or dissolving the nucleus pulposus. With respect to degenerative disc disease, the term therapeutically effective amount refers to the amount of chymopapain used to evacuate the nucleus pulposus to prepare the nuclear space in a disc for a nuclear replacement device or nuclear replacement material.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “immunogenic effective amount” of a chymopapain polypeptide disclosed herein refers to an amount of a polypeptide that is capable of inducing an immune response in an animal. The immune response can be determined by measuring a T or B cell response. Typically, the induction of an immune response is determined by the detection of antibodies specific for a chymopapain polypeptide.

As used herein, the term “immunogenic fragment thereof” refers to a fragment of a chymopapain polypeptide that is of a sufficient size to elicit an immune response in an animal. Typically, immunogenic fragments are at least 8 amino acids long and can include up to the full-length polypeptide. The immune response includes both a T and B cell response, but preferably is identified by the ability of the fragment to elicit antibodies.

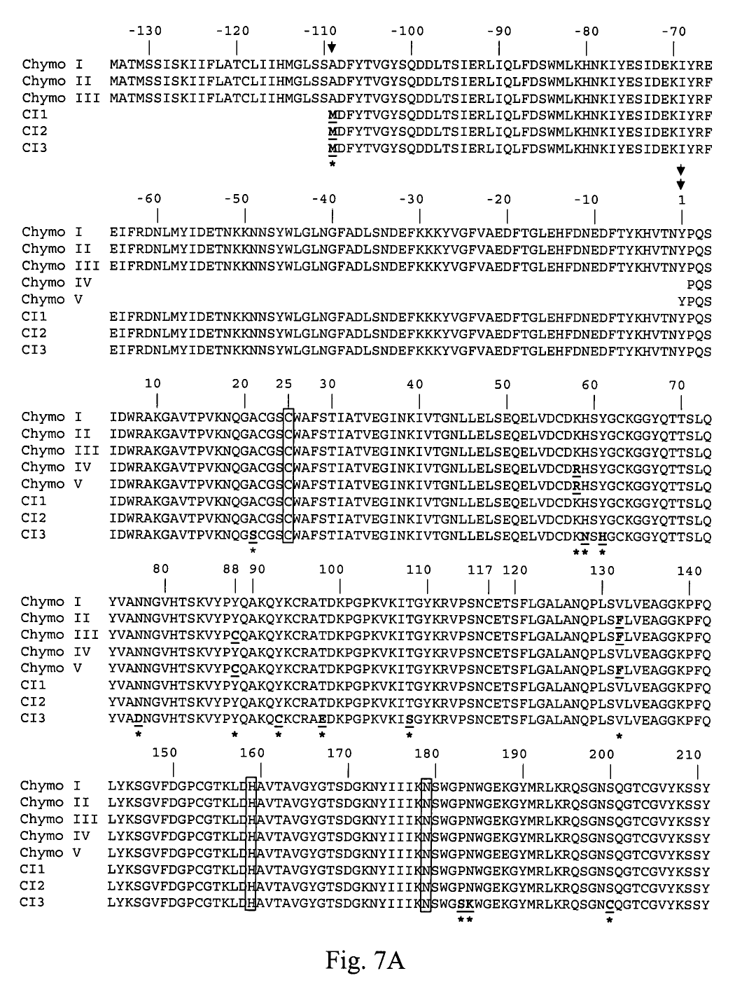

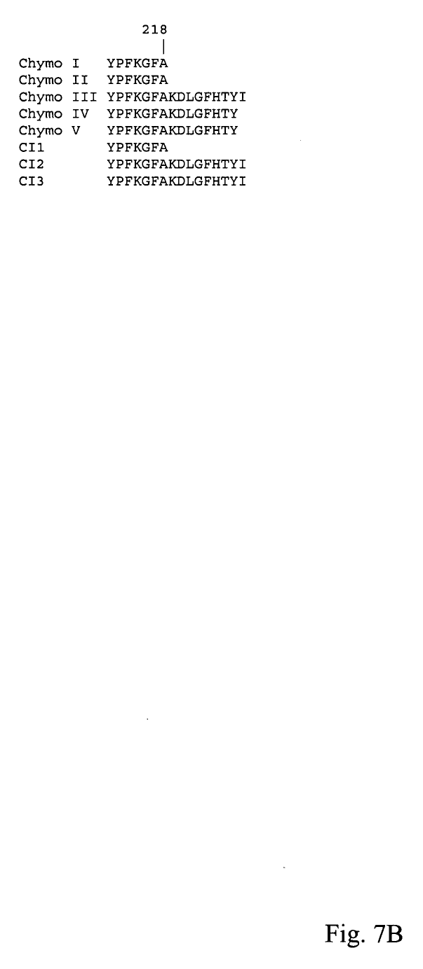

The term “binds specifically” refers to an antibody that binds particular chymopapain polypeptides and does not crossreact with other chymopapain isoenzymes or naturally occurring chymopapain, such as for example the chymopapains identified in FIGS. 7A and 7B. For example, an antibody that binds specifically to a chymopapain polypeptide such as CI3 does not bind to any of the other chymopapains shown in FIGS. 7A and 7B.

The term “epitope tagged” when used herein refers to a chimeric polypeptide comprising chymopapain linked to a “peptide tag”. The peptide tag has enough residues to provide an epitope against which an antibody can be made, yet is short enough such that it does not interfere with activity of the polypeptide to which it is fused. The peptide tag should be fairly unique so that the antibody does not substantially cross-react with other epitopes. Suitable peptide tags generally have at least six amino acid residues and usually between about 8 and 50 amino acid residues, preferably between about 10 and 20 amino acid residues. In an embodiment, chymopapain polypeptide is linked to a His tag comprising 6 or more histidine residues.

As used herein “fusion protein” refers to an additional polypeptide that is linked to a chymopapain, preferably at the N and/or C terminal terminus. The additional polypeptide can be a peptide tag that provides for ease of purification or identification, such as for example an epitope tag. In an embodiment, chymopapain is linked to a His tag comprising 6 or more histidine residues. Additional peptides or polypeptides can also be fused to enhance immunogenicity, such as bovine serum albumin, or keyhole lymphocyte hemocyanin.

### II. Modes for Carrying Out the Invention

At least 70% of chemonucleolysis patients do not require surgical intervention such as, for example, laminectomy, disectomy, microdiscectomy, or percutaneous discectomy (Waddell et al. “Surgical Treatment of Lumbar Disc Prolapse and Degenerative Lumbar Disc Disease.”Neck and Back Pain: The Scientific Evidence of Causes, Diagniosis and Treatment. Ed. Alf Nachemson and Egon Jonsson. Philadelphia: Lipincott Williams & Wilkins, 2000. 305-325.). However, patients with high serum levels of IgE antibodies against refined chymopapain are currently excluded from chemonucleolysis treatment. In these patients, antibodies to caricain and glycl endopeptidase account for approximately 75% of the total IgE antibodies against refined chymopapain (Dando et al., 1995, Spine, 20:981-985). Recombinant chymopapain and/or synthetic chymopapain could provide therapeutic benefits in these patients.

The present invention provides methods for administering recombinant chymopapain or synthetic chymopapain to treat a disc disorder including, but not limited to, increased intradiscal pressure, herniated nucleus pulposus, or nerve root compression or stretching. The recombinant or synthetic chymopapain can be prochymopapain or mature chymopapain. The recombinant or synthetic chymopapain can be naturally occurring or variant.

The present invention also provides novel recombinant or synthetic chymopapain isoenzymes. The chymopapain isoenzymes of the invention have at least about 80% amino acid identity to a reference sequence for chymopapain. The isoenzymes of the invention can be variants of prochymopapain or mature chymopapain. The isoenzymes of the invention can include substitution of one or more amino acid residues corresponding to amino acid residues 1, 130, 167, 168, 170, 186, 197, 202, 207, 217, 241, 292, 293, or 309 of a pro chymopapain reference sequence, such as any one of SEQ ID NO:16, 35, 36, 37, or 38. In an embodiment, the chymopapain isoenzymes of the invention include an amino acid sequence of SEQ ID NO:1, SEQ ID NO:3, or SEQ ID NO:5. The chymopapain isoenzymes of the invention are useful in methods for treating a disc disorder.

A. Chymopapain Isoenzymes

One aspect of the invention is chymopapain isoenzymes. The chymopapain isoenzymes of the invention are recombinant or synthetic. “Chymopapain isoenzyme” refers to a chymopapain that differs in amino acid sequence from a reference sequence for chymopapain. The reference sequence can be a prepro, pro, or mature form of chymopapain. The reference sequence can be a consequence sequence. Examples of reference sequences are shown in Table 1.

TABLE I

| | | | |
|---|---|---|---|
| | | | SEQ |
| | | | ID |
| Isoform | Form | Amino Acid Sequence | NO |

| | | | |
|---|---|---|---|
| Chymopapain | prepro | MATMSSISKIIFLATCLIIHMGLS | 7 |
| isoform I | | SADFYTVGYSQDDLTSIERLIQL | |
| (EMBL ac- | | FDSWMLKHNKIYESIDEKIYRE | |
| cession no. | | EIFRDNLMYIDETNKKNNSYW | |
| X97789) | | LGLNGFADLSNDEFKKKYVGF | |
| | | VAEDFTGLEHFDNEDFTYKHV | |
| | | TNYPQSIDWRAKGAVTPVKNQ | |
| | | GACGSCWAFSTIATVEGINKIV | |
| | | TGNLLELSEQELVDCDKHSYG | |
| | | CKGGYQTTSLQYVANNGVHTS | |
| | | KVYPYQAKQYKCRATDKPGPK | |
| | | VKITGYKRVPSNCETSFLGALA | |
| | | NQPLSVLVEAGGKPFQLYKSG | |
| | | VFDGPGGTKLDHAVTAVGYGT | |
| | | SDGKNYIIIKNSWGPNWGEKG | |
| | | YMRLKRQSGNSQGTCGVYKSS | |
| | | YYPFKGFA | |
| | pro | ADFYTVGYSQDDLTSIERLIQLF | 18 |
| | | DSWMLKHNKIYESDEKIYREE | |
| | | IFRDNLMYIDETNKKNNSYWL | |
| | | GLNGFADLSNDEFKKKYVGFV | |
| | | AEDFTGLEHFDNEDFTYKHVT | |
| | | NYPQSIDWRAKGAVTPVKNQG | |
| | | ACGSCWAFSTIATVEGINKIVT | |
| | | GNLLELSEQELVDCDKHSYGC | |
| | | KGGYQTTSLQYVANNGVHTSK | |
| | | VYPYQAKQYKCRATDKPGPKV | |
| | | KITGYKRVPSNCETSFLGALAN | |
| | | QPLSVLVEAGGKPFQLYKSGVF | |
| | | DGPCGTKLDHAVTAVGYGTSD | |
| | | GKNYIIIKNSWGPNWGEKGYM | |
| | | RLKRQSGNSQGTCGVYKSSYY | |
| | | PFKGFA | |
| | mature | YPQSIDWRAKGAVTPVKNQGA | 19 |
| | | CGSCWAFSTIATVEGINKIVTG | |
| | | NLLELSEQELVDCDKHSYGCK | |
| | | GGYQTTSLQYVANNGVHTSKV | |
| | | YPYQAKQYKCRATDKPGPKVK | |
| | | ITGYKRVPSNCETSFLGALANQ | |
| | | PLSVLVEAGGKPFQLYKSGVFD | |
| | | GPCGTKLDHAVTAVGYGTSDG | |
| | | KNYIIIKNSWGPNWGEKGYMR | |
| | | LKRQSGNSQGTCGVYKSSYYP | |
| | | FKGFA | |
| Chymopapain | prepro | MATMSSISKIIFLATCLIIHMGLS | 8 |
| isoform II | | SADFYTVGYSQDDLTSIERLIQL | |
| (EMBL ac- | | FDSWMLKHNKIYESIDEKIYRF | |
| cession no. | | EIFRDNLMYIDETNKKNNSYW | |
| AJ131995) | | LGLNGFADLSNDEFKKKYVGF | |
| | | VAEDFTGLEHFDNEDFTYKHV | |
| | | TNYPQSIDWRAKGAVTPVKNQ | |
| | | GACGSCWAFSTIATVEGINKIV | |
| | | TGNLLELSEQELVDCDKHSYG | |
| | | CKGGYQTTSLQYVANNGVHTS | |
| | | KVYPYQAKQYKCRATDKPGPK | |
| | | VKITGYKRVPSNCETSFLGALA | |
| | | NQPLSFLVEAGGKPFQLYKSGV | |
| | | FDGPCGTKLDHAVTAVGYGTS | |
| | | DGKNYIIIKNSWGPNWGEKGY | |
| | | MRLKRQSGNSQGTCGVYKSSY | |
| | | YPFKGFA | |
| | pro | ADFYTVGYSQDDLTSIERLIQLF | 20 |
| | | DSWMLKHNKIYESIDEKIYRFEI | |
| | | FRDNLMYIDETNKKNNSYWLG | |
| | | LNGFADLSNDEFKKKYVGFVA | |
| | | EDFTGLEHFDNEDFTYKHVTN | |
| | | YPQSIDWRAXGAVTPVKNQGA | |
| | | CGSCWAFSTIATVEGINKIVTG | |
| | | NLLELSEQELVDCDKHSYGCK | |
| | | GGYQTTSLQYVANNGVHTSKV | |
| | | YPYQAKQYKCRATDKPGPKVK | |
| | | ITGYKRVPSNCETSFLGALANQ | |
| | | PLSFLVEAGGKPFQLYKSGVFD | |
| | | GPCGTKLDHAVTAVGYGTSDG | |
| | | KNYIIIKNSWGPNWGEKGYMR | |
| | | LKRQSGNSQGTCGVYKSSYYP | |
| | | FKGFA | |
| | mature | YPQSIDWRAKGAVTPVKNQGA | 21 |
| | | CGSCWAFSTIATVEGINKIVTG | |
| | | NLLELSEQELVDCDKHSYGCK | |
| | | GGYQTTSLQYVANNGVHTSKV | |
| | | YPYQAKQYKCRATDKPGPKVK | |
| | | ITGYKRVPSNCETSFLGALANQ | |
| | | PLSFLVEAGGKPFQLYKSGVFD | |
| | | GPCGTKLDHAVTAVGYGTSDG | |
| | | KNYIIIKNSWGPNWGEKGYMR | |
| | | LKRQSGNSQGTCGVYKSSYYP | |
| | | FKGFA | |
| Chymopapain | prepro | MATMSSISKIIFLATCLIIHLMGLS | 9 |
| isoform III | | SADFYTVGYSQDDLTSIERLIQL | |
| (EMBL ac- | | FDSWMLKHNKIYESIDEKIYRF | |
| cession no. | | EIFRDNLMYIDETNKKNNSYW | |
| AJ131996) | | LGLNGFADLSNDEFKKKYVGF | |
| | | VAEDFTGLEHFDNEDFTYKHV | |
| | | TNYPQSIDWRAKGAVTPVKNQ | |
| | | GACGSCWAFSTIATVEGINKIV | |
| | | TGNLLELSEQELVDCDKHSYG | |
| | | CKGGYQTTSLQYVANNGVHTS | |
| | | KVYPCQAKQYKCRATDKPGPK | |
| | | VKITGYKRVPSNCETSFLGALA | |
| | | NQPLSFLVEAGGKPFQLYKSGV | |
| | | FDGPCGTKLDHAVTAVGYGTS | |
| | | DGKNYIIIKNSWGPNWGEKGY | |
| | | MRLKRQSGNSQGTCGVYKSSY | |
| | | YPFKGFAKDLGFHTYI | |
| | pro | ADFYTVGYSQDDLTSIERLIQLF | 22 |
| | | DSWMLKHNKIYESIDEKIYRFEI | |
| | | FRDNLMYIDETNKKNNSYWLG | |
| | | LNGFADLSNDEFKKKYVGFVA | |
| | | EDFTGLEHFDNEDFTYKHVTN | |
| | | YPQSIDWRAKGAVTPVKNQGA | |
| | | CGSCWAFSTIATVEGINKIVTG | |
| | | NLLELSEQELVDCDKHSYGCK | |
| | | GGYQTTSLQYVANNGVHTSKV | |
| | | YPCQAKQYKCRATDKPGPKVK | |
| | | ITGYKRVPSNCETSFLGALANQ | |
| | | PLSFLVEAGGKPFQLYKSGVFD | |
| | | GPCGTKLDHAVTAVGYGTSDG | |
| | | KNYIIIKNSWGPNWGEKGYMR | |
| | | LKRQSGNSQGTCGVYKSSYYP | |
| | | FKGFAKDLGFHTYI | |
| | mature | YPQSIDWRAKGAVTPVKNQGA | 23 |
| | | CGSCWAFSTIATVEGINKIVTG | |
| | | NLLELSEQELVDCDKHSYGCK | |
| | | GGYQTTSLQYVANNGVHTSKV | |
| | | YPCQAKQYKCRATDKPGPKVK | |
| | | ITGYKRVPSNCETSFLGALANQ | |
| | | PLSFLVEAGGKPFQLYKSGVFD | |
| | | GPCGTKLDHAVTAVGYGTSDG | |
| | | KNYIIIKNSWGPNWGEKGYMR | |
| | | LKRQSGNSQGTCGVYKSSYYP | |
| | | FKGFAKDLGFHTYI | |
| Chymopapain | mature | PQSIDWRAKGAVTPVKNQGAC | 10 |
| isoform IV | | GSCWAFSTIATVEGINKIVTGN | |
| (EMBL ac- | | LLELSEQELVDCDRHSYGCKG | |
| cession no. | | GYQTTSLQYVANNGVHTSKVY | |
| AJ131997) | | PYQAKQYKCRATDKPGPKVKI | |
| | | TGYKRVPSNCETSFLGALANQP | |
| | | LSVLVEAGGKPFQ | |
| Chymopapain | mature | YPQSIDWRAKGAVTPVKNQGA | 11 |
| isoform V | | CGSCWAFSTIATVEGINKIVTG | |
| (EMBL ac- | | NLLELSEQELVDCDRHSYGCK | |
| cession no. | | GGYQTTSLQYVANNGVHTSKV | |
| AJ131998) | | YPCQAKQYKCRATDKPGPKVK | |
| | | ITGYKRVPSNCETSFLGALANQ | |
| | | PLSFLVEAGGKPFQLYKSGVFD | |
| | | GPCGTKLDHAVTAVGYGTSDG | |
| | | KNYIIIKNSWGPNWGEEGYMR | |
| | | LKRQSGNSQGTCGVYKSSYYP | |
| | | FKGFAKDLGFHTY | |
| Consensus | | ADFYTVGYSQDDLTSIERLIQLF | |
| prochymopa- | | DSWMLKHNKIYESIDEKIYRFEI | |
| pain | | FRDNLMYIDETNKKNNSYWLG | |
| | | LNGFADLSNDEFKKKYVGFVA | |
| | | EDFTGLEHFDNEDFTYKHVTN | |
| | | YPQSIDWRAKGAVTPVKNQGA | |
| | | CGSCWAFSTIATVEGINKIVTG | |
| | | NLLELSEQELVDCDX₁HSYGCK | |
| | | GGYQTTSLQYVANNGVHTSKV | |
| | | YPX₂QAKQYKCRATDKPGPKV | |
| | | KITGYKRVPSNCETSFLGALAN | 16 |
| | | QPLSX₃LVEAGGKIPFQLYKSGV | |
| | | FDGPCGTKLDHAVTAVGYGTS | 35 |
| | | DGKNYIIIKNSWGPNWGEKGY | |
| | | MRLKRQSGNSQGTCGVYKSSY | 36 |
| | | YPFKGFAX₄X₅X₆X₇X₈X₉X₁₀X₁₁X₁₂ | |
| | | (1) When X₁ is K, X₂ is Y, | 37 |
| | | and X₃ is V, X₄-X₁₂ are ab- | |
| | | sent; or | |
| | | (2) When X₁ is K, X₂ is Y, | |
| | | and X₃ is F, X₄-X₁₂ are | |
| | | absent; or | |
| | | (3) When X₁ is K and X₂ is | 38 |
| | | C, X₃ is F, X₄ is K, X₅ is | |
| | | D, X₆ is L, X₇ is G, X₈ is | |
| | | F, X₉ is H, X₁₀ is T, X₁₁ is | |
| | | Y, and X₁₂ is I; or | |
| | | (4) When X₁ is R and X₂ is | |
| | | Y, X₃ is V, X₄ is K, X₅ is | |
| | | D, X₆ is L, X₇ is G, X₈ is | |
| | | F, X₉ is H, X₁₀ is T, X₁₁ is | |
| | | Y, and X₁₂ is absent; or | |
| | | (5) When X₁ is R and X₂ is | |
| | | C, X₃ is F, X₄ is K, X₅ is | |
| | | D, X₆ is L, X₇ is G, X₈ is | |
| | | F, X₉ is H, X₁₀ is T, X₁₁ is | |
| | | Y, and X₁₂ is absent | |
| Consensus | | YPQSIDWRAKGAVTPVKNQGA | |
| mature | | CGSCWAFSTIATVEGINKIVTG | |
| chymopapain | | NLLELSEQELVDCDX₁HSYGCK | |
| | | GGYQTTSLQYVANNGVHTSKV | |
| | | YPX₂QAKQYKCRATDKPGPKV | |
| | | KITGYKRVPSNCETSFLGALAN | |
| | | QPLSX₃LVEAGGKPFQLYKSGV | |
| | | FDGPCGTKLDHAVTAVGYGTS | 17 |
| | | DGKNYIIIKNSWGPNWGEKGY | |
| | | MRLKRQSGNSQGTCGVYKSSY | 39 |
| | | YPFKGFAX₄X₅X₆X₇X₈X₉X₁₀X₁₁X₁₂ | 40 |
| | | (1) When X₁ is K, X₂ is Y, | |
| | | and X₃ is V, X₄-X₁₂ are ab- | |
| | | sent; or | |
| | | (2) When X₁ is K, X₂ is Y, | 41 |
| | | and X₃ is F, X₄-X₁₂ are ab- | |
| | | sent; or | |
| | | (3) X₁ is K and X₂ is C, X₃ | 42 |
| | | is F, X₄ is K, X₅ is D, X₆ | |
| | | is L, X₇ is G, X₈ is F, X₉ | |
| | | is H, X₁₀ is T, X₁₁ is Y, | |
| | | and X₁₂ is I; or | |
| | | (4) When X₁ is R and X₂ is | |
| | | Y, X₃ is V, X₄ is K, X₅ is | |
| | | D, X₆ is L, X₇ is G, X₈ is | |
| | | F, X₉ is H, X₁₀ is T, X₁₁ is | |
| | | Y, and X₁₂ absent; or | |
| | | (5) When X₁ is R and X₂ is | |
| | | C, X₃ is F, X₄ is K, X₅ is | |
| | | D, X₆ is L, X₇ is G, X₈ is | |
| | | F, X₉ is H, X₁₀ is T, X₁₁ is | |
| | | Y, and X₁₂ is absent | |

“Chymopapain isoenzyme” specifically encompasses modifications of the reference sequence, and naturally occurring chymopapain variants. When the chymopapain isoenzyme is a naturally occurring variant of the reference sequence, the isoenzyme is designated “a naturally occurring chymopapain variant.” Chymopapain isoenzymes of the invention include naturally occurring variants isolated from nature. In an embodiment, the isoenzymes of the invention are isolated from a bromeliad such as, for example, papaya and pineapple.

The chymopapain isoenzymes of the invention can include deletions and additions of amino acids, as well as amino acid substitutions. Variants of naturally occurring prochymopapain or mature chymopapain described herein, can be made, for example, using any of the techniques and guidelines for conservative and non-conservative mutations set forth, for example, in U.S. Pat. No. 5,364,934. Chymopapain isoenzymes can be generated by any known method for substituting, deleting, or inserting one or more codons that result in a change in the amino acid sequence of chymopapain as compared with a reference sequence for chymopapain.

A chymopapain isoenzyme of the present invention has at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity to a chymopapain reference sequence. In an embodiment, the chymopapain isoenzyme includes an amino acid sequence of SEQ ID NO: 1, 3, 5, 24, or 25. In an embodiment the isoenzyme has cysteine protease activity. In an embodiment, a chymopapain isoenzyme of the invention has a lower antigenicity than refined chymopapain.

A chymopapain isoenzyme of the invention can be a variant of prochymopapain. In an embodiment, the isoenzyme includes substitution of one or more amino acid residues corresponding to amino acid residues 1, 130, 167, 168, 170, 186, 197, 202, 207, 217, 241, 292, 293, or 309 of SEQ ID NO:16, 35, 36, 37, or 38, wherein the isoenzyme has at least about at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity to an amino acid sequence of SEQ ID NO:16, 35, 36, 37, or 38. Preferred substitutions include A1M, A130S, K167R, H168N, Y170H, N186D, Y197C Y202C, T207E, T217S, V241F, P292S, N293K, and/or S309C. In an embodiment, the mature form of the isoenzyme has cysteine-protease activity.

In an embodiment, the isoenzyme is a variant of prochymopapain and includes an amino acid sequence of SEQ ID NO:1 or SEQ ID NO:3. In another embodiment, the isoenzyme is a variant of prochymopapain variant and includes amino acid substitutions at residues corresponding to residues 1, 130, 168, 170, 186, 202, 207, 217, 292, 293, and 309 of SEQ ID NO:16, 35, 36, 37, or 38. In a further embodiment, the isoenzyme includes an amino acid sequence of SEQ ID NO:5.

A chymopapain isoenzyme of the invention can be a variant of mature chymopapain. In an embodiment, the isoenzyme includes substitution of one or more of amino acid residues corresponding to amino acid residues 21, 58, 59, 61, 77, 88, 93, 98, 108, 132, 183, 184, or 200 of SEQ ID NO:17, 39, 40, 41, or 42, wherein the isoenzyme has at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity to an amino acid sequence of SEQ ID NO: 17, 39, 40, 41, or 42. Preferred substitutions include A21S, K58R, H59N, Y61H, N77D, Y88C, Y93C, T98E, T108S, V132F, P183N, N184K, and/or S200C. In an embodiment, the isoenzyme has cysteine protease activity.

In an embodiment, the isoenzyme is a variant of mature chymopapain and includes amino acid substitutions at residues corresponding to residues 21, 59, 61, 77, 93, 98, 108, 183, 184, and 200 of SEQ ID NO:17, 39, 40, 41, or 42. In another embodiment, the isoenzyme is a variant of mature chymopapain and includes an amino acid sequence of SEQ ID NO:25.

The chymopapain isoenzymes of the invention can have cysteine protease activity. In an embodiment, the chymopapain isoenzymes of the invention have cysteine protease activity equal to or greater than the cysteine protease activity of naturally occurring chymopapain, such as for example, chymopapain comprising an amino acid sequence of SEQ ID NO:10, SEQ ID NO:11, SEQ ID NO:19, SEQ ID NO:21, or SEQ ID NO: 23. Cysteine protease activity can be detected with a substrate by detecting release of a chromogenic or fluorogenic leaving group. A number of cysteine protease substrates that contain a chromogenic or fluorogenic leaving group are known in the art and commercially available, including for example N-α-benzoyl-DL-arginine p-nitroanilide (BAPNA) which contains a chromogenic p-nitroaniline leaving group, benzyloxycarbonyl-Phe-Arg-AMC which contains a fluorogenic 7-amino-4-methylcoumarin (AMC) leaving group, and Ac-Leu-Thr-Phe-Lys-ACC which contains a fluorogenic 7-amino-4-carbamoylmethylcoumarin (ACC) leaving group. Assays for detecting cysteine protease activity typically employ a spectrophotometer or fluorimeter to detect release of the chromogenic or fluorogenic leaving group from the substrate. A number of assays are known in the art for detecting cysteine protease activity. See, for example, Taylor et al., 1999, Plant Sci., 145:41-47; Selzer et al., 1999, Proc. Natl. Acad. Sci., 96:11015-11022; Harris et al., 2000, Proc. Natl. Acad. Sci., 97:7754-7769; and U.S. Pat. No. 4,719,108. In an embodiment, cysteine protease activity is detected with a BAPNA assay as described in the Examples.

Chymopapain isoenzymes of the invention can be generated by any known methods of substituting, deleting, or inserting of one or more codons encoding chymopapain. Guidance in determining which amino acid residue can be inserted, substituted or deleted without adversely affecting the desired activity can be found by comparing the sequence of chymopapain with that of homologous known protein molecules and minimizing the number of amino acid sequence changes made in regions of high homology. In an embodiment, reference sequences for chymopapain are aligned and compared to identity regions of high and low homology. Functional domains can also be identified in chymopapain that have homology to known polypeptides. In an embodiment, the amino acid sequence of chymopapain is compared to the amino acid sequence of another papaya cysteine protease such as, for example, papain.

Functional domains of chymopapain are known. A crystal structure for chymopapain has been described, for example, in Maes et al., 1996, Biochem., 35:16292-16298. The basic fold of chymopapain is very similar to that of other papaya cysteine proteases. The fold is comprised of an L domain (residues 10-111 and 211-218; numbering corresponding to mature chymopapain; see FIGS. 7A and 7B) and an R domain (residues 1-9 and 112-210; numbering corresponding to mature chymopapain; see FIGS. 7A and 7B) (Maes et al., 1996, Biochem., 35:16292-16298). The L domain is α-helical. The R domain consists of extensive antiparallel β-sheet interactions. The active site is located at the interface between the L and R domains. The active site is composed of residues 24-42 in the L domain (numbering corresponding to mature chymopapain; see FIGS. 7A and 7B), with the active center Cys 25 positioned at the start of the α-helix (Maes et al., 1996, Biochem., 35:16292-16298). The catalytic triad is formed by Cys25 in the L domain and His159 and Asn179 in the R domain.

In an embodiment, one or more residues in the L domain and/or R domain are deleted or substituted. In the L domain, one or more of residues 21, 58, 59, 61, 77, 88, 93, 98, 108 can be substituted. Preferred substitutions include A21S, K58R, H59N, Y61H, N77D, Y88C, Y93C, T98E, and T108S. In the R domain, one or more of residues 132, 183, 184, and 200 can be substituted. Preferred substitutions include V132F, P183S, N184K, and S200C. In an embodiment, one or more residues in the active site are deleted and/or substituted. In an embodiment, deletions and/or substitutions in the L domain, R domain, and/or active site increase the cysteine protease activity of the chymopapain isoenzyme as compared to mature chymopapain, such as, SEQ ID NO:10, SEQ ID NO:11, SEQ ID NO:17, SEQ ID NO:19, SEQ ID NO:21, SEQ ID NO: 23, 39, 40, 41, or 42.

The S1 subsite, formed by Cys25, Gly23, and Gly65, and the S2 subsite, formed by residues 67, 68, 69, 133, 157, and 207 (numbering corresponding to mature chymopapain; see FIGS. 7A and 7B), are believed to determine substrate specificity (Maes et al., 1996, Biochem., 35:16292-16298). In an embodiment, one or more residues in the S1 and/or S2 subsite are deleted or substituted. In an embodiment, deletions and/or substitutions in the S1 subsite and/or S2 site increase the binding affinity of the chymopapain isoenzyme for glycosoaminoglycans as compared to mature chymopapain, such as, SEQ ID NO:10, SEQ ID NO:11, SEQ ID NO:17, SEQ ID NO:19, SEQ ID NO:21, SEQ ID NO: 23, SEQ ID NO:39, 40, 41, or 42. In an embodiment, chymopapain isoenzymes of the invention are specific for glycosoaminoglycans in the nucleus pulposus and do not hydrolyze collagen in the annulus fibrosus.

The sequences of the functional domains can be compared and aligned to other known sequences of cysteine proteases that can be provided by GenBank (accessible on the internet at www-ncbi-nln-nih-gov), and locations of amino acid positions for substitutions can be identified as those positions that show a high degree of variability in amino acids, i.e. at least 3 different amino acids are found at that position when different sequences are aligned and compared or have a lower percentage of sequence identity i.e. less than 90% sequence identity. When sequences are aligned, the positions that show variability can either have conservative amino acid substitutions or non-conservative amino acid substitutions. If the position has conservative amino acid substitutions, that would indicate that the amino acid substituted at that position should be of the same type as those observed to be at that position in naturally occurring proteins. For examples of such substitutions, see Table 2. In particular embodiments, conservative substitutions of interest are shown in Table 2 under the heading of preferred substitutions.

TABLE 2

| Original | | Preferred |
|---|---|---|
| Residue | Exemplary Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; norleucine | |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; type | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

Amino acid substitutions can be the result of replacing one amino acid with another amino acid having similar structural and/or chemical properties, such as the replacement of a leucine with a serine, i.e., conservative amino acid replacements. Insertions or deletions can optionally be in the range of about 1 to 5 amino acids. The variation allowed can be determined by systematically making insertions, deletions or substitutions of amino acids in the sequence and testing the resulting variants for activity exhibited by the full-length or mature naturally occurring sequence. Preferably, variants have a biological activity of the source molecule, such as for example, cysteine protease activity.

The variations can be made using methods known in the art such as oligonucleotide-mediated (site-directed) mutagenesis, alanine scanning, and PCR mutagenesis. Site-directed mutagenesis (Carter et al., 1986, Nucl. Acids Res., 13:4331; Zoller et al., 1987, Nucl. Acids Res., 10:6487), cassette mutagenesis (Wells et al., 1985, Gene, 34:315), restriction selection mutagenesis (Wells et al., 1986, Philos. Trans. R. Soc. London SerA, 317:415) or other known techniques can be performed on the cloned DNA to produce the chymopapain variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant (Cunningham and Wells, 1989, Science, 244: 1081-1085). Alanine is also typically preferred because it is the most common amino acid. Further, it is frequently found in both buried and exposed positions (Creighton, The Proteins, (W.H. Freeman & Co., N.Y.); Chothia, 1976, J. Mol. Biol., 150:1). If alanine substitution does not yield adequate amounts of variant, an isoteric amino acid can be used.

Chymopapain isoenzymes of the invention also include naturally occurring polypeptide variants. A reference chymopapain can be obtained from a single source of C. papaya. Chymopapain variants can be identified using antibodies that specifically bind chymopapain, by sequence identity to a chymopapain reference sequence, such as any one of SEQ ID NO:7-11, 18-23, or 35-42, or hybridization of probe to a polynucleotide encoding a chymopapain variant under stringent conditions, moderately stringent conditions, or highly stringent conditions.

Chymopapain isoenzyme fragments are provided herein. A chymopapain isoenzyme can be a fragment of a naturally occurring chymopapain or variant of a naturally occurring chymopapain. Such fragments can be truncated at the N-terminus or C-terminus, or can lack internal residues, for example, when compared with a full-length protein. Certain fragments can lack amino acid residues that are not essential for a desired biological activity of the chymopapain isoenzyme.

The invention also contemplates immunogenic fragments of chymopapain isoenzymes. Immunogenic fragments are at least 8 amino acids in length, more preferably 8-50 amino acids, more preferably at least 10 amino acids, and more preferably at least 20 amino acids up to a full-length polypeptide. Immunogenic fragments can be prepared synthetically, recombinantly, or by enzymatic digestion of chymopapain isoenzyme. Immunogenic fragments can be predicted by analyzing the primary amino acid sequence of chymopapain using commercially available services such as Epipredict or Epitope informatics or publicly available programs such as are available.

Chymopapain isoenzyme fragments can be prepared by any of a number of conventional techniques. Desired peptide fragments can be chemically synthesized. An alternative approach involves generating chymopapain polypeptide fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. In an embodiment, chymopapain isoenzyme fragments share at least one biological and/or immunological activity with chymopapain polypeptide having an amino acid sequence of SEQ ID NO:1, SEQ ID NO:3, SEQ ID NO:5, SEQ ID NO:7, SEQ ID NO:8, SEQ ID NO:, SEQ ID NO:9, SEQ ID NO:10, SEQ ID NO:11, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:35, SEQ ID NO:36, SEQ ID NO:37, SEQ ID NO:38, SEQ ID NO:39, SEQ ID NO:40, SEQ ID NO:41, or SEQ ID NO:42. In an embodiment, the biological activity is cysteine protease activity.

Chymopapain isoenzymes of the invention or fragments thereof can be pegylated (e.g. one or more polyethylene glycol (PEG) groups are conjugated to C-amino group(s) of a chymopapain isoenzyme of the invention) to reduce the immunogenicity of the isoenzyme or fragment thereof. Pegylation can increase in vivo half-life and/or reduce immunogenicity and potential toxicity of therapeutic proteins (Abuchowski et al., 1977, J. Biol. Chem., 252:3582-86). Methods for pegylating therapeutic proteins are well known in the art and described, for example, in U.S. Pat. Nos. 4,179,337 and 6,136,563.

B. Vectors, Host Cells, and Recombinant Methods

The chymopapain isoenzymes of the invention are produced by synthetic and recombinant methods. Accordingly, a second aspect of the invention relates to polynucleotides encoding the isoenzymes of the invention, recombinant vectors, and host cells containing the recombinant vectors, as well as methods of making the vectors and host cells by recombinant methods.

Isolation of DNA encoding chymopapain isoenzymes of the invention or the sequence of DNA encoding naturally occurring chymopapain can be obtained from a genomic library of sequences isolated from Carica papaya leaves (Taylor et al., 1999, Plant Sci., 145:41-47).

Libraries can be screened with probes (such as antibodies to chymopapain polypeptides or oligonucleotides of at least about 20-80 bases) designed to identify the gene of interest or the protein encoded by it. Screening the DNA or genomic library with the selected probe can be conducted using standard procedures, such as described in Sambrook et al, Molecular Cloning: A Laboratory Manual (New York: Cold Spring Harbor Laboratory Press, 1989). An alternative means to isolate the gene encoding chymopapain is to use PCR methodology [Sambrook et al., supra; Dieffenbach et al., PCR Primer: A Laboratory Manual (Cold Spring Harbor Laboratory Press, 1995)]. For example, PCR primers for chymopapain isoenzymes described herein are shown in Example 1.

“Oligonucleotides” are short-length, single- or double-stranded polydeoxynucleotides that are chemically synthesized by known methods such as, for example, phosphotriester, phosphite, or phosphoramidite chemistry, using solid phase techniques such as those described in EP Pat. Pub. No. 266,032 published May 4, 1988, or via deoxynucleoside H-phosphonate intermediates as described by Froehler et al., 1986, Nucl. Acids Res., 14: 5399-5407. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Polynucleotides having protein coding sequence can be obtained by screening selected DNA or genomic libraries using the deduced amino acid sequence disclosed herein, and, if necessary, using conventional primer extension procedures as described in Sambrook et al., supra, to detect precursors and processing intermediates of mRNA that may not have been reverse-transcribed into DNA.

Isolated nucleic acids comprising polynucleotides encoding chymopapain can be identified by searching C. papaya genomic sequences for cysteine protease motifs. Once putative chymopapain sequences have been identified, PCR primers can be designed as shown in Example 1 to amplify the desired polynucleotide sequences. Once amplified, the polynucleotide molecule can be isolated and/or purified and cloned into a recombinant vector.

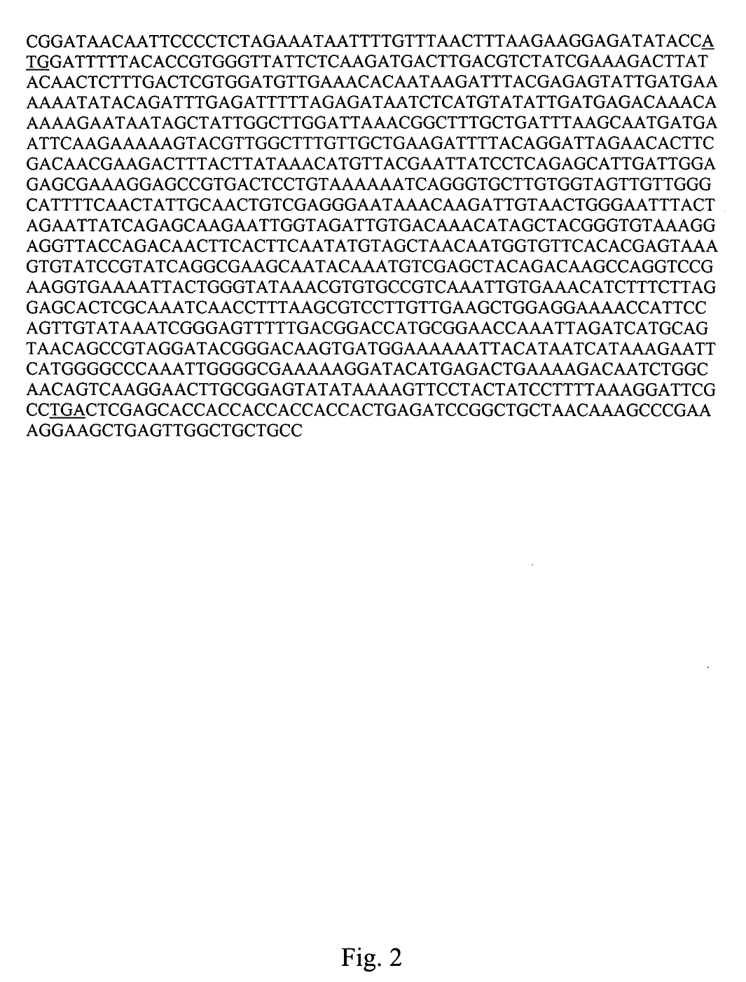

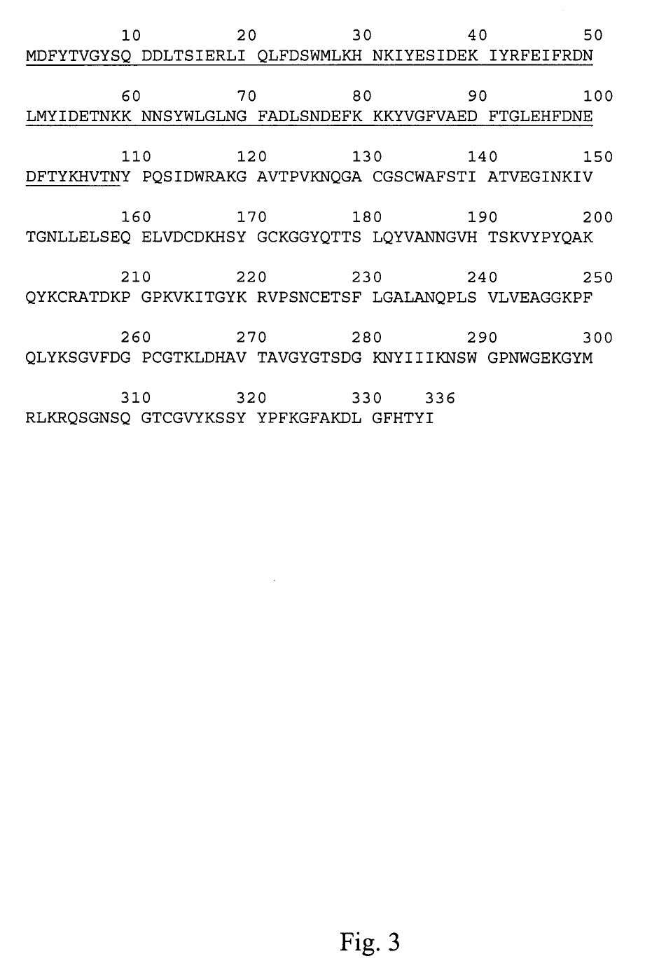

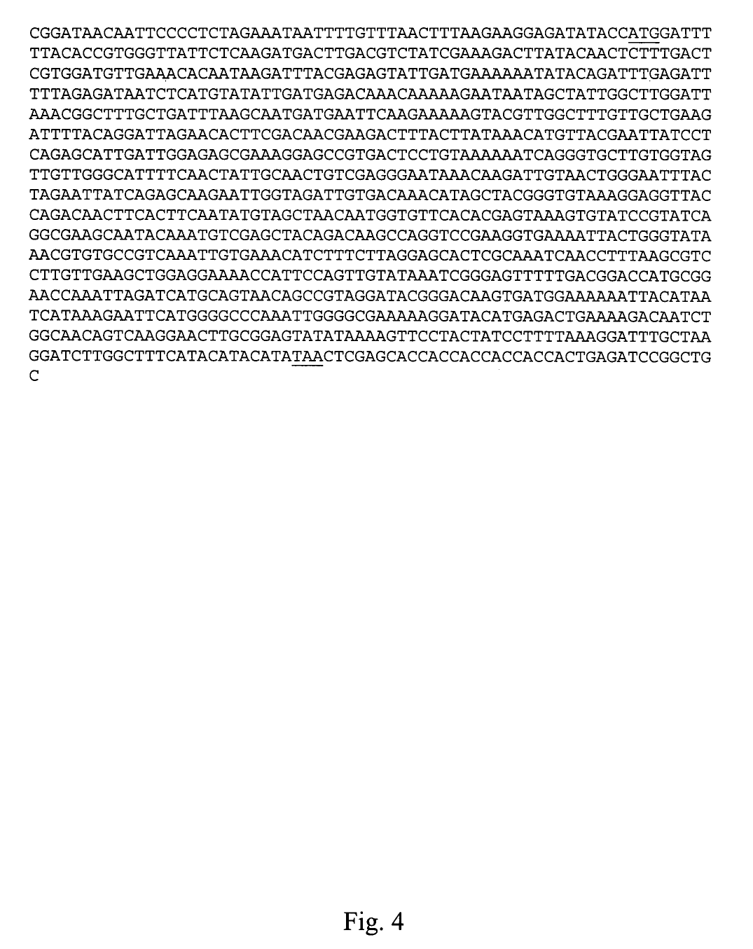

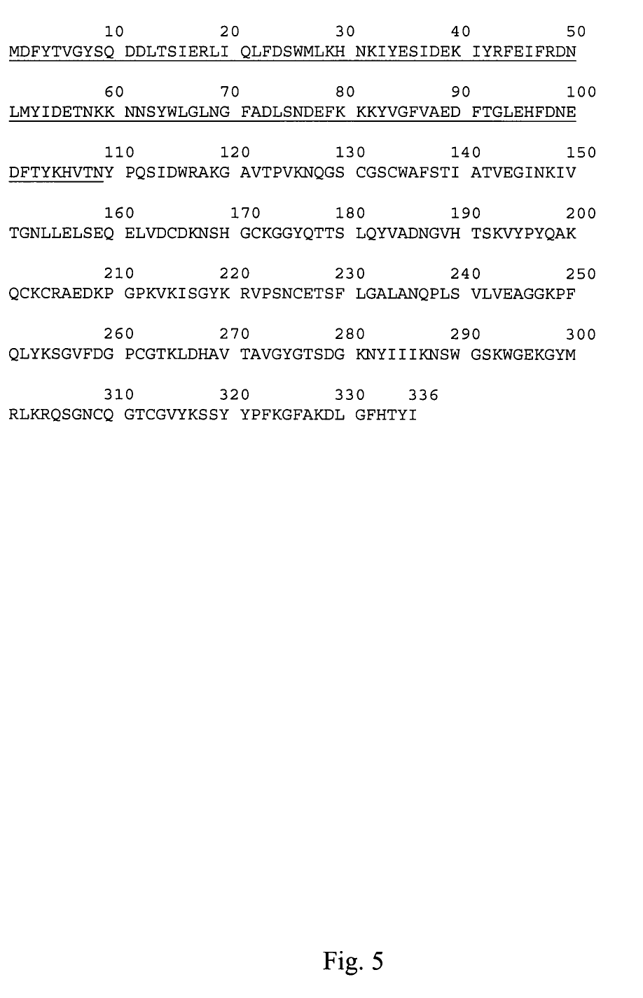

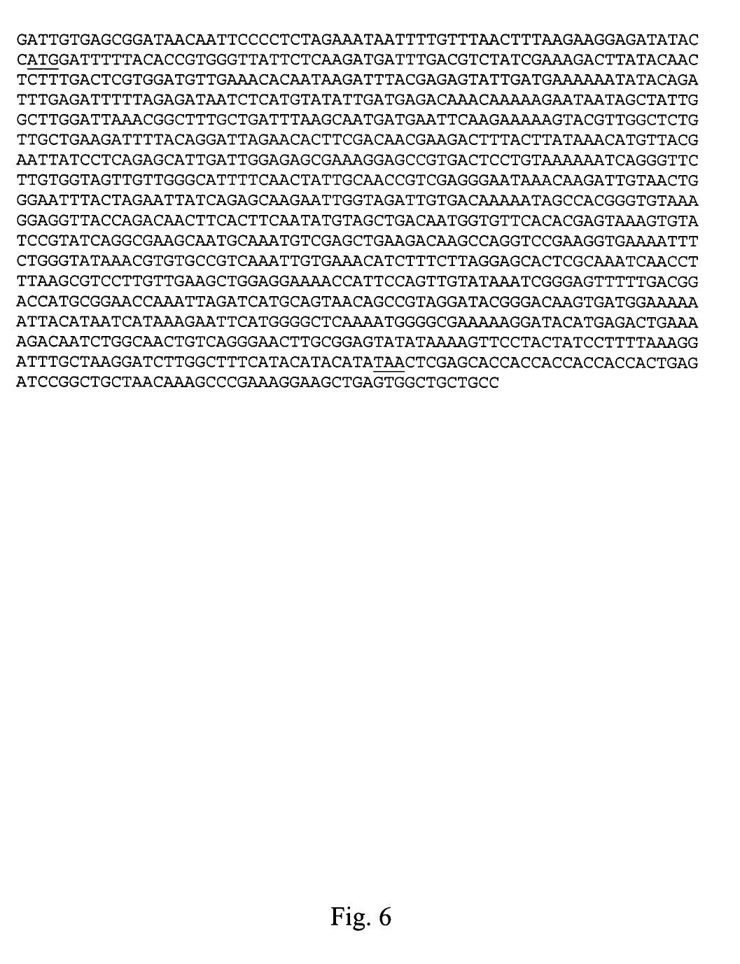

The invention also includes variants of nucleic acid molecules encoding chymopapain. In one embodiment, the invention includes polynucleotides having at least about 70% sequence identity, more preferably about 75% sequence identity, more preferably about 80% sequence identity, more preferably about 85% sequence identity, more preferably about 90% sequence identity, more preferably about 91% sequence identity, more preferably about 92% sequence identity, more preferably about 93% sequence identity, more preferably about 94% sequence identity, more preferably about 95% sequence identity, more preferably about 96% sequence identity, more preferably about 97% sequence identity, more preferably about 98% sequence identity, more preferably about 99% sequence identity to a nucleotide sequence encoding any of the amino acid sequences of SEQ ID NOs:1, 3, 5, 7, 8, 9, 10, 11, 16, 17, 18, 19, 20, 21, 22, 23, 24, 25, 35, 36, 37, 38, 39, 40, 41, or 42 or a nucleotide sequence complementary to full length sequences. In an embodiment, the polynucleotides include a nucleic acid sequence of SEQ ID NO:2 (FIG. 2), SEQ ID NO:4 (FIG. 4), or SEQ ID NO:6 (FIG. 6).

The chymopapain isoenzymes of the invention can be synthesized or prepared by techniques well known in the art. See, for example, Creighton, Proteins: Structures and Molecular Principles, W. H. Freeman & Co., New York, N.Y. (1983), which is incorporated herein by reference in its entirety. Polynucleotide sequences encoding the chymopapain isoenzymes of the invention can be synthesized, and/or cloned, and expressed according to techniques well known to those of ordinary skill in the art. See, for example, Sambrook, et al., Molecular Cloning, A Laboratory Manual, Vols. 1-3, Cold Spring Harbor Press, Cold Spring Harbor, N.Y. (1989).

The polynucleotides of the invention can be produced by standard recombinant methods known in the art, such as polymerase chain reaction (Sambrook et al., Molecular Cloning, A Laboratory Manual, Vols. 1-3, Cold Spring Harbor Press, Cold Spring Harbor, N.Y. (1989). The peptide constructs can be assembled from polymerase chain reaction cassettes sequentially cloned into a vector containing a selectable marker for propagation in a host. Such markers include dihydrofolate reductase or neomycin resistance for eukaryotic cell culture and tetracycline or ampicillin resistance genes for culturing in E. coli and other bacteria.

Bacterial expression vectors can be used to express chymopapain isoenzymes of the invention. Plasmid pET 28a (Novagen, Madison, Wis.) is an example of a suitable expression vector. The nucleotide sequence and map of the pET 28a vector is known and readily available on the internet at www-emdbiosciences-com. Baculovirus expression vectors can be used to express chymopapain isoenzymes of the invention. Many baculovirus expression systems are commercially available, such as Baculogold (Pharmingen), Bac-n-Blue (Invitrogen), Bac Pak (Clontech), and Bac Vector (Novagen, Madison, Wis.). For example, insect cells can be co-transfected with baculovirus transfer vector comprising DNA encoding chymopapain and AcMNPV baculovirus DNA. Methods for producing cysteine proteases using baculovirus expression vectors are described, for example, in Pechan et al., 2004, Protein Exp. Purif., 34:134-141, Mason et al., 2001, Protein Exp. Purif., 23:45-54, and Vernet et al., 1990, J. Biol. Chem., 265:16661-16666.

Representative examples of appropriate hosts include, but are not limited to, bacterial cells such as E. coli, Streptornyces and Salmonella typheriuni, fungal cells, yeast such as Pichia pectoris; insect cells such as Drosophilia S2 and Spodoptera Sf9 or Sf21, animal cells such as CHO, COS, and Bowes melanoma cells, and plant cells. Appropriate culture medium and conditions for the above-described host cells are known in the art.

The polynucleotides of the invention can be operably linked to an appropriate promoter, such as the isopropyl β-D-thiogalactopyranoside (IPTG) inducible T7 promoter in plasmid pET 28a (Studier et al., 1990, Methods Enzymol., 185:60-89. Other suitable promoters are known in the art. The expression constructs may further contain sites for transcription initiation, transcription termination, and a ribosome binding site for translation. The coding portion of the mature polypeptide expressed by the constructs can include a translation initiating codon at the beginning and a termination codon (UAA, UGA, or UAG) appropriately positioned at the end of the polypeptide to be translated.

Introduction of the recombinant vector into the host cell can be effected by calcium phosphate transfection, DEAE-dextran mediated transfection, cationic lipid-mediated transfection, electroporation, transduction, infection, or other methods. Such methods are described in standard laboratory manuals such as Sambrook, et al., 1989, Molecular Cloning, A Laboratory Manual, Vols. 1-3, Cold Spring Harbor Press, Cold Spring Harbor, N.Y. or Davis et al., 1986, Basic Methods in Molecular Biology. Commercial transfection reagents, such as Lipofectamine (Invitrogen, Carlsbad, Calif.) and FuGENE 6™ (Roche Diagnostics, Indianapolis, Ind.), are also available.

C. Purification

Chymopapain isoenzymes of the invention can be recovered from culture medium or from host cell lysates. If membrane-bound, it can be released from the membrane using a suitable detergent solution or by enzymatic cleavage. One example of a suitable detergent is X-100. Cells employed in expression of chymopapain isoenzymes can be disrupted by various physical or chemical means, such as freeze-thaw cycling, sonication, mechanical disruption, or cell lysing agents.

It may be desired to purify chymopapain isoenzymes of the invention from other recombinant cell proteins. The following procedures are exemplary of suitable purification procedures: fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; and metal chelating columns to bind epitope-tagged forms of the chymopapain isoenzymes. Various methods of protein purification can be employed and such methods are known in the art and described for example in Deutscher, 1990, Methods in Enzymology, 182 and Scopes, Protein Purification: Principles and Practice, Springer-Verlag, New York (1982). “Purified” proteins are preferably those that have been purified to homogeneity as detected by SDS-PAGE and staining with Coomasie Blue. The purification step(s) selected will depend, for example, on the nature of the production process used and the particular chymopapain produced.

Chymopapain isoenzymes of the invention can be expressed in a modified form, such as a fusion protein, and can include secretion signals and/or additional heterologous functional regions. For example, a region of additional amino acids can be added to the N-terminus or C-terminus of the polypeptide to facilitate detection or purification, or improve persistence in the host cell during, for example, purification or subsequent handling and storage. Examples of additional amino acids include peptide tags that can be added to the polypeptide to facilitate detection and/or purification. Such peptide tags include, but are not limited to, H is, HA, Avi, biotin, c-Myc, VSV-G, HSV, V5, or FLAG™.

Chymopapain isoenzymes of the invention can be recovered and purified from recombinant cell cultures by methods known in the art, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. In an embodiment, high performance liquid chromatography (HPLC) is employed for purification.

D. Antibodies

The present invention further provides anti-chymopapain antibodies. Exemplary antibodies include polyclonal and monoclonal antibodies. In an embodiment, an anti-chymopapain antibody of the invention binds specifically to a chymopapain isoenzyme of the invention. Methods for determining whether a particular antibody is specific for a particular polypeptide and does not cross react with others is known to those of skill in the art, and include competitive binding assays.

1. Polyclonal Antibodies

The anti-chymopapain antibodies can include polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent can include the chymopapain polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants that can be employed include Freund's complete adjuvant and MPL-TDM adjuvant (monophosphoryl Lipid A, synthetic trehalose dicorynomycolate).

2. Monoclonal Antibodies

The anti-chymopapain antibodies may, alternatively, be monoclonal antibodies. Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include a chymopapain isoenzyme or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Coding, Monoclonal Antibodies: Principles and Practice, Academic Press, (1986) pp. 59-1031).

The culture medium in which the hybridoma cells are cultured can then be assayed for the presence of monoclonal antibodies directed against chymopapain. In an embodiment, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, 1980, Anal. Biochem., 107:220.

E. Compositions

The chymopapain isoenzymes of the invention can be used in compositions that are useful, for example, in chemonucleolysis. The compositions of the invention include recombinant or synthetic prochymopapain isoenzymes and/or recombinant or synthetic mature chymopapain isoenzymes. The compositions of the invention can include one or more forms of chymopapain, for example, SEQ ID NO:1, 3, 5, 7, 8, 9, 10, 11, 18, 19, 20, 21, 22, 23, 24, or 25. The chymopapain isoenzymes can be naturally occurring or variant chymopapain. In an embodiment, the compositions of the invention include chymopapain having an amino sequence of SEQ ID NO:24 and/or SEQ ID NO:25. Compositions of the invention can contain additional proteases such as, for example, papain and/or chondrotinase. In an embodiment, the additional proteases are recombinant or synthetic. In an embodiment, the compositions of the invention do not contain caricain, and/or glycl endopeptidase.

The compositions of the invention can be lyophilized or in soluble form. The compositions of the invention can also include a carrier. The use of buffers, stabilizers, reducing agents, anti-oxidants and chelating agents in the preparation of protein based compositions, particularly pharmaceutical compositions, is well known in the art. See, Wang et al., “Review of Excipients and pHs for Parenteral Products Used in the United States.” J. Parent. Drug Assn., 34(6):452-462 (1980); Wang et al., “Parenteral Formulations of Proteins and Peptides: Stability and Stabilizers,” J. Parent. Sci. and Tech., 42:S4-S26 (Supplement 1988); Lachman, et al., “Antioxidants and Chelating Agents as Stabilizers in Liquid Dosage Forms-Part 1,” Drug and Cosmetic Industry, 102(1): 36-38, 40 and 146-148 (1968); Akers, M. J., “Antioxidants in Pharmaceutical Products,” J. Parent. Sci. and Tech., 36(5):222-228 (1988); and Methods in Enzymology, Vol. XXV, Colowick and Kaplan eds., “Reduction of Disulfide Bonds in Proteins with Dithiothreitol,” by Konigsberg, pages 185-188.

Suitable carriers include pharmaceutically acceptable carriers, excipients, or stabilizers which are nontoxic to the cell or mammal being exposed thereto at the dosages and concentrations employed. Often the physiologically acceptable carrier is sterile water or an aqueous pH buffered solution. Suitable physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ polyethylene glycol (PEG), and PLURONICS™. In an embodiment, chymopapain isoenzymes of the invention are suspended in sterile water containing cysteine.

The composition can include one or more preservatives such as phenol, cresol, paraminobenzoic acid, BDSA, sorbitrate, chlorhexidine, benzalkonium chloride, or the like. Suitable stabilizers include carbohydrates such as threlose or glycerol. The composition can include a stabilizer such as one or more of microcrystalline cellulose, magnesium stearate, mannitol, sucrose to stabilize, for example, the physical form of the composition; and one or more of glycine, arginine, hydrolyzed collagen, or protease inhibitors to stabilize, for example, the chemical structure of the composition. Suitable suspending agents include carboxymethyl cellulose, hydroxypropyl methylcellulose, hyaluronic acid, alginate, chonodroitin sulfate, dextran, maltodextrin, dextran sulfate, or the like. The composition can include an emulsifier such as polysorbate 20, polysorbate 80, pluronic, triolein, soybean oil, lecithins, squalene and squalanes, sorbitan treioleate, or the like. The composition can include an antimicrobial such as phenylethyl alcohol, phenol, cresol, benzalkonim chloride, phenoxyethanol, chlorhexidine, thimerosol, or the like. Suitable thickeners include natural polysaccharides such as mannans, arabinans, alginate, hyaluronic acid, dextrose, or the like; and synthetic ones like the PEG hydrogels of low molecular weight and aforementioned suspending agents.

In an embodiment, the compositions of the invention are formulated with cysteine, such as for example sodium cysteinate hydrochloride. Compositions of the invention can be formulated with a reducing agents, antioxidants, and/or chelating agents. Suitable reducing agents, which maintain the reduction of reduced cysteines, include dithiothreitol (DTT also known as Cleland's reagent) or dithioerythritol at 0.01% to 0.1% wt/wt; acetylcysteine or cysteine at 0.1% to 0.5% (pH 2-3); and thioglycerol at 0.1% to 0.5% (pH 3.5 to 7.0) and glutathione. See Akers, supra, at pages 225 to 226. Suitable antioxidants include sodium bisulfite, sodium sulfite, sodium metabisulfite, sodium thiosulfate, sodium formaldehyde sulfoxylate, and ascorbic acid. See, for example, Akers, supra, at pages 225. Suitable chelating agents, which chelate trace metals to prevent the trace metal catalyzed oxidation of reduced cysteines, include citrate, tartarate, ethylenediaminetetraacetic acid (EDTA) in its disodium, tetrasodium, and calcium disodium salts, and diethylenetriamine pentaacetic acid (DTPA). See, for example, Wang, supra, and Akers, supra.

Compositions of the invention can have a pH of about 5 to about 7.5. In an embodiment, compositions of the invention can have a pH of about 5 to about 6. In an embodiment, compositions of the invention can have a pH of about 5.1, 5.2, 5.3, 5.4, 5.5, 5.6, 5.7, 5.8, or 5.9

The isoenzymes of the invention can be administered parenterally, including percutaneously or through a surgical incision using infusion techniques, in dosage unit formulations containing conventional non-toxic pharmaceutically acceptable carriers, adjuvants or vehicles. Compositions of the invention can be in the form sterile injectable preparations, such as sterile injectable aqueous or oleagenous suspensions. Methods for formulating a pharmaceutical composition are generally known in the art. A thorough discussion of formulation and selection of pharmaceutically acceptable carriers, stabilizers, and isomolytes can be found in Remington's Pharmaceutical Sciences (18^{th} ed.; Mack Publishing Company, Eaton, Pa., 1990), herein incorporated by reference.

The agent of the present invention can also be formulated in a sustained-release form to prolong the presence of chymopapain in the treated mammal, generally for longer than one day. Many methods of preparation of a sustained-release formulation are known in the art and are disclosed in Remington's Pharmaceutical Sciences (18^{th} ed.; Mack Publishing Company, Eaton, Pa., 1990), herein incorporated by reference. For administration as injectable solutions or suspensions, the compositions can be formulated according to techniques well-known in the art, using suitable dispersing or wetting and suspending agents, such as sterile oils, including synthetic mono- or diglycerides, and fatty acids, including oleic acid.

Dosage unit forms suitable for use in dissolving or treating a nucleus pulposus contain about 10 to about 100,000 units of chymopapain. In an embodiment, the dosage unit form includes about 100 to about 10,000 units of chymopapain. In an embodiment, the dosage unit form includes about 300 to about 8,000 units of chymopapain. In an embodiment, the dosage unit form includes about 500 to about 5,000 units of chymopapain. In an embodiment, the dosage unit form includes about 2,000 to about 4,000 BAPNA units of chymopapain. BAPNA units of chymopapain can be determined by the BAPNA assay described in the Examples and/or U.S. Pat. No. 5,468,480. A dosage unit form can contain about 0.1 to about 10 mg of chymopapain. In an embodiment, a dosage unit form includes about 0.5 to about 8 mg chymopapain. In an embodiment, a dosage unit form includes about 1 to about 5 mg of chymopapain. In an embodiment, a dosage unit form includes about 1, 1.25, 1.5, 1.75, 2, 2.5, 3, 3.5, 4, 4.5, or 5 mg of chymopapain. In an embodiment, a dosage unit form includes about 2.5, 2.6, 2.7, 2.8, 2.9, 3, 3.1, 3.2, 3.3, 3.4, or 3.5 mg of chymopapain.

The chymopapain isoenzymes of the invention can be employed in immunogenic compositions. Immunogenic compositions are useful to elicit chymopapain antibodies. These antibodies are useful, for example, for purifying chymopapain from C. papaya or cell culture. The immunogenic composition includes recombinant or synthetic chymopapain isoenzymes or immunogenic fragments thereof. The composition can include one or more isoenzymes of chymopapain, for example, SEQ ID NO: 1, SEQ ID NO:3, SEQ ID NO: 5, SEQ ID NO:10, SEQ ID NO: 11, SEQ ID NO:18, SEQ ID NO:19, SEQ ID NO:20, SEQ ID NO:21, SEQ ID NO:22, SEQ ID NO:23, SEQ ID NO:24, and SEQ ID NO:25. The isoenzymes can be naturally occurring or variant chymopapain. In an embodiment, the composition includes a chymopapain isoenzyme having an amino sequence of SEQ ID NO:24 and/or SEQ BD NO:25.

In an embodiment, the chymopapain isoenzymes are present in an immunogenic effective amount. An immunogenic effective amount is an amount of chymopapain that induces an immune response in an animal. The actual amount of chymopapain can vary depending on the animal to be immunized, the route of administration and adjuvants. Immunogenic dosages can be determined by those of skill in the art. The immune response may be indicated by T and/or B cell responses. Typically, the immune response is detected by the presence of antibodies that specifically bind to a particular chymopapain isoenzymes such as, for example SEQ ID NO:10, SEQ ID NO: 11, SEQ ID NO:19, SEQ ID NO:21, SEQ ID NO:23, SEQ ID NO:24, or SEQ ID NO:25. Methods of detecting antibodies to chymopapain are known to those of skill in the art and include such assays as ELISA assays, western blot assays, and competition assays.

In one embodiment, animals are immunized against the antigen, immunogenic conjugates, or derivatives by combining, e.g., 100 μg or 5 μg of the protein or conjugate with 3 volumes of Freund's complete adjuvant and injecting the solution intradermally at multiple sites. One month later the animals are boosted with ½ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

F. Uses and Methods

The chymopapain isoenzymes of the invention described herein have many applications including, for example, in wound treatment, chemonucleolysis, development of anti-chymopapain antibodies, food additives, and additives for detergents.

Recombinant or synthetic chymopapain is useful, for example, in opthalmology for the treatment of eye lesions and removal of the vitreous humor of the eye (vitroectomy) and for debridement of eschar tissues of burns, ulcers, pressure necroses, bed sores, and other wounds in which devitalized tissues are present. Compositions for such uses are typically provided in a form suitable for topical application such as, for example, sterile solutions, gels, suspensions, and ointments that can be applied directly to the wound or via a wound dressing impregnated with the chymopapain or a composition thereof (see, for example, U.S. Pat. No. 5,468,480). The recombinant or synthetic chymopapain can be naturally occurring chymopapain and/or chymopapain isoenzymes of the invention.

Recombinant or synthetic chymopapain is useful in chemonucleolysis methods for treating patients that have a disc disorder or a disease or disorder secondary to the disc disorder. Examples of a disc disorder include, but are not limited to, increased intradiscal pressure in an intervertebral spinal disc, sciatica, a herniated or extruded intervertebral spinal disc, scoliosis, nerve root compression or stretching, and degenerative disc disease. Examples of a disease or disorder secondary to a disc disorder include, but are not limited to, herniated disc secondary to increased intradiscal pressure, nerve root compression or stretching secondary to herniated disc, or sciatica secondary to nerve root compression or stretching. In an embodiment, a disc having increased intradiscal pressure stretches, compresses, or causes compression or stretching of the adjacent nerve root. If the disc is a lumbar disc, compression or stretching of the adjacent nerve root can cause sciatica. In an embodiment, a patient with sciatica has one or more of the following indications: leg pain worse than low back pain; nerve root impingement at clinically suspected level demonstrated by MRI, CT, or myelography; objective neurologic deficit (e.g., diminished DTR, motor weakness, or hypalgesia in dermatomal distribution); and/or radicular symptoms reproduced by sciatic stretch tests.

The methods of the invention include locally administering recombinant chymopapain and/or synthetic chymopapain or a composition thereof to a disc. The chymopapain can be a naturally occurring chymopapain or a chymopapain isoenzyme of the invention described herein. The chymopapain can be prochymopapain or mature chymopapain. The composition can include one or more chymopapain such as, for example, SEQ ID NO: 1, 3, 5, 7, 8, 9, 10, 11, 18, 19, 20, 21, 22, 23, 24, or 25. The chymopapain may be naturally occurring or variant chymopapain. In an embodiment, the composition includes a chymopapain isoenzyme having an amino acid sequence of SEQ ID NO: 1, SEQ ID NO:3, SEQ ID NO:5, SEQ ID NO:24 and/or SEQ ID NO:25.

In an embodiment, locally administering includes contacting the nucleus pulposus of the disc with recombinant chymopapain and/or synthetic chymopapain or compositions thereof. In an embodiment, contacting includes inserting a delivery device into the nucleus pulposus, confirming placement of the delivery device with an imaging device, and delivering the composition into the nucleus pulposus. See, for example, U.S. Pat. Nos. 4,719,108 and 5,468,480. In an embodiment, the chymopapain is delivered into the nucleus pulposus by injection. Preferably the nucleus pulposus is dissolved. The delivery device can be inserted into the nucleus pulposus percutaneously or through a surgical incision such as, for example, during a spinal surgical procedure. The delivery device can be, for example, a canula, endoscope with operating channel, needle, or catheter attached to a syringe. The delivery device can be manually operated or operated by or with the assistance of a computer, such as for example robotics. The imaging device can be, for example, x-ray, ultrasound, computerized topography (CT) scanner, or magnetic resonance imager (MRI).

In one aspect, the invention relates to methods of reducing intradiscal pressure in an intervertebral spinal disc with recombinant or synthetic chymopapain or compositions thereof. Intradiscal pressure can increase due to inflammation or trauma to the spine, and can be accompanied by protruded or extended disc, degenerative disc, herniated lumbar disc disease, and/or chronic low back pain. Intradiscal pressure can be reduced by locally administering recombinant or synthetic chymopapain to the disc. In an embodiment, the patient has a documented herniated nucleus pulposus that is unresponsive to conservative care measures including, but not limited to, bed rest, exercise, anti-inflammatory drugs, body corset, epidural blocks, physical therapy, and traction. In an embodiment, a disc having increased intradiscal pressure compresses, stretches, or causes compression or stretching of the adjacent nerve root. If the disc is a lumbar disc, compression or stretching of the adjacent nerve root can cause sciatica. In an embodiment, the method includes contacting the nucleus pulposus in a disc that has increased intradiscal pressure with recombinant or synthetic chymopapain or a composition thereof. Preferably the nucleus pulposus is dissolved. The disc can be a thoracic, cervical, or lumbar disc. In an embodiment, the disc is a cervical or lumbar disc.

In another aspect, the invention relates to methods for treating a herniated or extruded intervertebral spinal disc with recombinant or synthetic chymopapain or compositions thereof. The herniated or extruded disc can be associated with inflammation, trauma to the spine, sciatica, herniated lumbar disc disease, chronic low back pain, and/or nerve root compression or stretching. In an embodiment, the patient has a documented herniated nucleus pulposus that is unresponsive to conservative care measures including, but not limited to, bed rest, exercise, anti-inflammatory drugs, body corset, epidural blocks, physical therapy, and traction. The herniated or extruded disc can be a thoracic, cervical, or lumbar disc. In an embodiment, the herniated or extruded disc is a cervical or lumbar disc. In an embodiment, the method includes contacting the nucleus pulposus of a disc at the level of the herniation or extrusion with recombinant or synthetic chymopapain or a composition thereof. In an embodiment, the nucleus pulposus is dissolved.

In another aspect, the invention relates to methods of hydrolyzing and/or removing the nucleus pulposus from the nuclear space of an intervertebral disc with recombinant or synthetic chymopapain or a composition thereof. In an embodiment, the nucleus pulposus is removed in preparation for insertion of a nucleus replacement device including, but not limited to, a prosthetic disc nucleus implant such as, for example, PDN-SOLO® (Raymedica, Minneapolis, Minn.), a polymeric hydrogel such as, for example, polyvinyl (PVA) hydrogel or carageenan pyrrolidone (PVP) hydrogel (Stryker Howmedica Osteonic, Allendale, N.J.; Replication Medical, New Brunswick, N.J.), an elastomeric device such as, for example, a polycarbonate urethane coil (Sulzer Spine-Tech, Edina, Minn.), an injectable protein hydrogel that hardens inside the nuclear space (Cryolife, Kennesaw, Ga.), an injectable thermopolymer that hardens inside the nuclear space such as, for example, polyurethane (Disc Augmentation Technologies), a polymeric balloon (Disc Dynamics, Eden Prairie, Minn.), or a polymeric prosthetic disc device such as, for example, PRODISC® (Spine Solutions GmbH, Tuttlingen, Germany), Link SB Charite (Waldeman Link GmbH and Co., Hamburg, Germany), MAVERICK™ (Medtronic Sofamor Danek, Memphis, Tenn.), and FLEXICORE® (Spinecore, Summit, N.J.). In an embodiment, the method includes contacting a nucleus pulposus to be removed with recombinant or synthetic chymopapain or a composition thereof. Preferably the nucleus pulposus is removed by dissolving the nucleus pulposus. The disc can be a thoracic, cervical, or lumbar disc. In an embodiment, the disc is a cervical or lumbar disc.

In another aspect, the invention relates to methods for treating spinal stiffness and/or increasing spinal flexibility. Chemonucleolysis can provide a minimally invasive procedure for anterior spinal release in scoliosis correction, resulting in a less stiff and more flexible spine at the abnormal curvature associated with scoliosis that facilitates external bracing. In an embodiment, the method includes contacting the nucleus pulposus of a disc at the level of the curvature with recombinant or synthetic chymopapain or a composition thereof. In an embodiment, the disc or discs are in a region of the spine that has abnormal curvature associated with scoliosis. See, for example, U.S. Pat. Nos. 4,719,108 and 5,468,480. Preferably the nucleus pulposus is dissolved.

In another aspect, the invention relates to methods of decreasing intracapsular pressure in a prostate associated with benign prostate hypertrophy. In an embodiment, the method includes locally administering recombinant or synthetic chymopapain or a composition thereof to a prostate. In an embodiment, locally administering includes delivering chymopapain into the capsule of the prostate.

In another aspect, the invention relates to methods of debulking a tumor prior to surgical removal. In an embodiment, the method includes locally administering recombinant or synthetic chymopapain or a composition thereof to a tumor. In an embodiment, locally administering includes delivering chymopapain into the tumor with a canula, endoscope with operating channel, catheter, or needle. In an embodiment, the tumor is a solid tumor.

The chymopapain isoenzymes of the invention can be used to develop anti-chymopapain antibodies. The anti-chymopapain antibodies of the invention can be used to purify recombinant chymopapain from the supernatant of a cell. In an embodiment, the recombinant chymopapain includes an amino acid sequence of SEQ ID NO:1, 3, 5, 24, or 25.

The chymopapain isoenzymes of the invention can be used as a food additive, such as for example a meat tenderizer or clarifier, or an additive for detergents, such as for example, in detergents for cleaning laundry, contact lenses, or dentures. In an embodiment, the chymopapain isoenzyme includes an amino acid sequence of SEQ ID NO:1, 3, 5, 24, or 25.

### EXAMPLES

The present invention may be better understood with reference to the following examples. These examples are intended to be representative of specific embodiments of the invention, and are not intended as limiting the scope of the invention.

### Example 1

### Identification of Novel Chymopapain Isoenzymes from a cDNA Library

Chymopapain, a cysteine proteinase, is useful in chemonucleolysis for the treatment of a herniated nucleus pulposus. A cDNA library was constructed from mRNA isolated from the leaves of C. papaya to identify novel chymopapain isoenzymes.

a) Construction of cDNA Library

Messenger RNA (mRNA) was isolated from C. papaya leaves using an mTRAP mRNA isolation kit (Active Motif, Carlsbad, Calif.) according to the manufacturer's instructions. A cDNA library was synthesized from the isolated mRNA using a ZAP Express XR Library Construction Kit (Stratagene, La Jolla, Calif.) according to the manufacturer's instructions. The cDNA library was amplified by polymerase chain reaction (PCR; Sambrook et al., Molecular Cloning A Laboratory Manual, Cold Spring Harbor, 1989) using Pfu Turbo DNA polymerase (Stratagene, La Jolla, Calif.). The following primers were used to amplify the chymopapain cDNAs:

| | | | | |
|---|---|---|---|---|
| Chymopapain | 5′ | Forward | 5′TTGTTGTTCC | SEQ ID |
| isoform I | prochymo | | ATGGATTTTTA | NO: 26 |
| | | | CACCGTGGGTT | |
| | | | ATTCTTC3′ | |
| | 3′ CHYM | Reverse | 5′TTGTTGTTCT | SEQ ID |
| | HIS | | CGAGAGCAAA | NO: 27 |
| | | | ACCTTTAAAAG | |
| | | | GATAATATG3′ | |
| | 3′ CHYM | Reverse | 5′TTGTTGTTCT | SEQ ID |
| | | | CGAGTCAAGCA | NO: 28 |
| | | | AAACCTTTAAA | |
| | | | AGGATAATATG | |
| | | | 3′ | |
| Chymopapain | 5′ | Forward | 5′TTGTTGTTCC | SEQ ID |
| isoform II | prochymo | | ATGGATTTTTA | NO: 29 |
| | | | CACCGTGGGTT | |
| | | | ATTCTTC3′ | |
| | 3′ | Reverse | 5′TTGTTGTTCT | SEQ ID |
| | short 95 | | CGAGGGCGAAT | NO: 30 |
| | prochymo | | CCTTTAAAAGG | |
| | HIS | | ATAGTAG3′ | |
| | 3′ | Reverse | 5′TTGTTGTTCT | SEQ ID |
| | short 95 | | CGAGTCAGGCG | NO: 31 |
| | prochymo | | AATCCTTTAAA | |
| | | | AGGATAGTAG3′ | |
| Chymopapain | 5′ | Forward | 5′TTGTTGTTCC | SEQ ID |
| isoforms III, | prochymo | | ATGGATTTTTA | NO: 32 |
| IV, and V | | | CACCGTGGGTT | |
| | | | ATTCTTC3′ | |
| | 3′ long | Reverse | 5′TTGTTGTTCT | SEQ ID |
| | prochymo | | CGAGTATGTAT | NO: 33 |
| | HIS | | GTATGAAAGCC | |
| | | | AAGATCC3′ | |
| | 3′ long | Reverse | 5′TTGTTGTTCT | SEQ ID |
| | prochymo | | CGAGTTATATG | NO: 34 |
| | | | TATGTATGAAA | |
| | | | GCCAAGATCC3′ | |

The primers were designed to introduce unique restriction sites upstream of the start codon (Nco I) and downstream of the stop codon (Xho I) for cloning into an E. coli expression vector. Primers designated “HIS” contained a nucleic acid sequence encoding a C-terminal hexa-histidine tag to facilitate purification. The His peptide tag binds to a metal chelating column such as, for example, a Ni-NTA column.

b) Selection of Transformants

The cDNAs were subcloned into pET 28a expression vectors (Novagen) which contain an isopropyl β-D-thiogalactopyranoside (IPTG) inducible T7 RNA polymerase/promoter system developed by Studier et al., 1990, Methods Enzymol., 185:60-89. Competent E. coli were transformed with the cloned expression vector (Sambrook et al., Molecular Cloning A Laboratory Manual, Cold Spring Harbor, 1989). Transformants were selected by plating the cells on medium containing kanamycin. Plasmid DNA was isolated from the transformed cells with a miniprep kit (Qiagen, Valencia, Calif.). The purified plasmid DNA was digested with restriction enzymes Nco I and Xho I (Promega, Madison, Wis.) and the cloned chymopapain cDNA insert was verified by gel electrophoresis (Sambrook et al., Molecular Cloning A Laboratory Manual, Cold Spring Harbor, 1989).

c) Sequencing of cDNAs

Expression vectors identified as positive for chymopapain cDNA inserts were sequenced with an ABI 377 automated DNA fragment analyzer (Applied Biosystems, Foster City, Calif.). Amino acid sequences of the cDNAs were deduced and aligned with the amino acid sequences of the five chymopapain isoforms identified by Taylor et al., 1999, Plant Sci., 145:41-47 (FIGS. 7A and 7B). Three novel chymopapain isoenzymes, CI1, CI2, and CI3, were identified. The amino acid sequences of the pro and mature forms of the identified chymopapain isoenzymes are shown in Table 3.

TABLE 3

| | | | |
|---|---|---|---|
| Chymopapain | | SEQ ID | |
| Isoenzyme | Amino Acid Sequence | NO: | Figure |

| | | | | |
|---|---|---|---|---|
| CI1 | Pro | MDFYTVGYSQDDLTSIER | 1 | 1 |
| | form | LIQLFDSWMLKHNKIYESI | | |
| | | DEKIYRFEIFRDNLMYIDE | | |
| | | TNKKNNSYWLGLNGFAD | | |
| | | LSNDEFKKKYVGFVAEDF | | |
| | | TGLEHFDNEDFTYKHVTN | | |
| | | YPQSIDWRAKGAVTPVKN | | |
| | | QGACGSCWAFSTIATVEGI | | |
| | | NKIVTGNLLELSEQELVDC | | |
| | | DKHSYGCKGGYQTTSLQ | | |
| | | YVANNGVHTSKVYPYQA | | |
| | | KYKCRATDKPGPKVKITG | | |
| | | YKRVPSNCETSFLGALAN | | |
| | | QPLSVLVEAGGKPFQLYK | | |
| | | SGVFDGPCGTKLDHAVTA | | |
| | | VGYGTSDGKNYIIIKNSW | | |
| | | GPNWGEKGYMRLKRQSG | | |
| | | NSQGTCGVYKSSYYPFKG | | |
| | | FA | | |
| | Mature | YPQSIDWRAKGAVTPVKN | 19 | |
| | form | QGACGSCWAFSTIATVEGI | | |
| | | NKIVTGNLLELSEQELVDC | | |
| | | DKHSYGCKGGYQTTSLQ | | |
| | | YVANNGVHTSKVYPYQA | | |
| | | KYKCRATDKPGPKVKITG | | |
| | | YKRVPSNCETSFLGALAN | | |
| | | QPLSVLVEAGGKPFQLYK | | |
| | | SGVFDGPCGTKLDHAVTA | | |
| | | VGYGTSDGKNYIIIKNSW | | |
| | | GPNWGEKGYMRLKRQSG | | |
| | | NSQGTCGVYKSSYYPFKG | | |
| | | FA | | |
| CI2 | Pro | MDFYTVGYSQDDLTSIER | 3 | 3 |
| | form | LIQLFDSWMLKHNKIYESI | | |
| | | DEKIYRFEIFRDNLMYIDE | | |
| | | TNKKNNSYWLGLNGFAD | | |
| | | LSNDEFKKKYVGFVAEDF | | |
| | | TGLEHFDNEDFTYKHVTN | | |
| | | YPQSIDWRAKGAVTPVKN | | |
| | | QGACGSCWAFSTIATVEGI | | |
| | | NKIVTGNLLELSEQ | | |
| | | ELVDCDKHSYGCKGGYQ | | |
| | | TTSLQYVANNGVHTSKVY | | |
| | | PYQAKQYKCRATDKPGP | | |
| | | KVKITGYKRVPSNCETSFL | | |
| | | GALANQPLSVLVEAGGKP | | |
| | | FQLYKSGVFDGPCGTKLD | | |
| | | HAVTAVGYGTSDGKNYIII | | |
| | | KNSWGPNWGEKGYMRL | | |
| | | KRQSGNSQGTCGVYKSSY | | |
| | | YPFKGFAKDLGFHTYI | | |
| | Mature | YPQSIDWRAKGAVTPVKN | 24 | |
| | form | QGACGSCWAFSTIATVEGI | | |
| | | NKIVTGNLLELSEQELVDC | | |
| | | DKHSYGCKGGYQTTSLQ | | |
| | | YVANNGVHTSKVYPYQA | | |
| | | KQYKCRATDKPGPKVKIT | | |
| | | GYKRVPSNCETSFLGALA | | |
| | | NQPLSVLVEAGGKPFQLY | | |
| | | KSGVFDGPCGTKLDHAVT | | |
| | | AVGYGTSDGKNYIIIKNS | | |
| | | WGPNWGEKGYMRLKRQS | | |
| | | GNSQGTCGVYKSSYYPFK | | |
| | | GFAKDLGFHTYI | | |
| CI3 | Pro | MDFYTVGYSQDDLTSIER | 5 | 5 |
| | form | LIQLFDSWMLKHNKIYESI | | |
| | | DEKIYRFEIFRDNLMYIDE | | |
| | | TNKKNNSYWLGLNGFAD | | |
| | | LSNDEFKKKYVGFVAEDF | | |
| | | TGLEHFDNEDFTYKHVTN | | |
| | | YPQSIDWRAKGAVTPVKN | | |
| | | QGSCGSCWAFSTIATVEGI | | |
| | | NKIVTGNLLELSEQELVDC | | |
| | | DKNSHGCKGGYQTTSLQ | | |
| | | YVADNGVHTSKVYPYQA | | |
| | | KQCKCRAEDKPGPKVKIS | | |
| | | GYKRVPSNCETSFLGALA | | |
| | | NQPLSVLVEAGGKPFQLY | | |
| | | KSGVFDGPCGTKLDHAVT | | |
| | | AVGYGTSDGKNYIIIKNS | | |
| | | WGSKWGEKGYMRLKRQS | | |
| | | GNCQGTCGVYKSSYYPFK | | |
| | | GFAKDLGFHTYI | | |
| | Mature | YPQSIDWRAKGAVTPVKN | 25 | |
| | form | QGSCGSCWAFSTIATVEGI | | |
| | | NKIVTGNLLELSEQELVDC | | |
| | | DKNSHGCKGGYQTTSLQ | | |
| | | YVADNGVHTSKVYPYQA | | |
| | | KQCKCRAEDKPGPKVKIS | | |
| | | GYKRVPSNCETSFLGALA | | |
| | | NQPLSVLVEAGGKPFQLY | | |
| | | KSGVFDGPCGTKLDHAVT | | |
| | | AVGYGTSDGKNYIIIKNS | | |
| | | WGSKWGEKGYMRLKRQS | | |
| | | GNCQGTCGVYKSSYYPFK | | |
| | | GFAKDLGFHTYI | | |

Compared to the pro from of chymopapain I (SEQ ID NO:18), the pro form of CI1 has an amino acid substitution at residue 1, the pro form of CI2 has an amino acid substitution at residue 1 and nine additional amino acids at the C-terminus, and the pro form of CI3 has amino acid substitutions at residues 1, 130, 168, 170, 186, 202, 207, 217, 292, 293, and 309 and nine additional amino acids at the C-terminus. The absence of the prepeptide sequence in the novel isoenzymes is a consequence of the primers used for amplification of the chymopapain coding sequences. The primers hybridized to the portion of the nucleic acid sequence encoding the propeptide sequence, which is downstream from the nucleotides encoding the prepeptide sequence.

d) Expression of Novel Chymopapain Isoenzymes

The chymopapain isoenzymes of the invention were expressed in E. coli cells. The cells were transformed with pET 28a expression vectors containing cDNA inserts encoding the chymopapain isoenzymes as described above. The cells were grown at 37° C. in LB broth containing 50 μg/ml kanamycin. Cultures were incubated until an OD₆₀₀ₙₘ of 0.25 was reached and then induced with IPTG. Cells were harvested 3 hr post induction. Cells were lysed in 1% SDS, centrifuged to remove debris, and the supernatants were analyzed by SDS-PAGE (Sambrook et al., Molecular Cloning A Laboratory Manual, Cold Spring Harbor, 1989). Total protein was isolated from cells containing expression vectors coding for CI2 and CI3 before and after IPTG induction. Cells transformed with expression vectors coding for CI2 or CI3 contained more total protein after IPTG induction as compared to a control, demonstrating the transformed cells were producing the chymopapain isoenzymes (data not shown).

### Example 2

### Expression of Novel Chymopapain Isoenzymes in Insect Cells

The chymopapain isoenzymes described in Example 1 were produced in a baculovirus expression system. Many baculovirus expression systems are commercially available, such as Baculogold (Pharmingen), Bac-n Blue (Invitrogen), Bac Pak (Clontech), and Bac Vector (Novagen).

Protein Expression

Methods for producing cysteine proteases in a baculovirus expression system are described in Pechan et al., 2004, Protein Exp. Purif, 34:134-141 and Mason et al., 2001, Protein Exp. Purif, 23: 45-54, and Vernet et al., 1990, J. Biol. Chem., 265:16661-16666. The novel isoenzymes described in example 1 were produced utilizing the BacPak6 baculovius expression system (Clontech) according to the manufacturer's instructions. Briefly, cDNA encoding the prepro form CI1, CI2, or CI3 described in Example 1 was subcloned into a baculovirus transfer vector PACGP67. Spodoptera frugiperda (Sf) 21 cells were co-transfected with purified baculovirus transfer vector and AcNPV baculovirus DNA. Resultant baculovirus were amplified in Sf21 cells and virus titers determined. Sf21 cells were then infected with virus at a multiplicity of infection between 0.1 and 10 pfu/cell (Vernet et al., 1990, J. Biol. Chem., 265:16661-16666). The infected cells were grown in suspension.

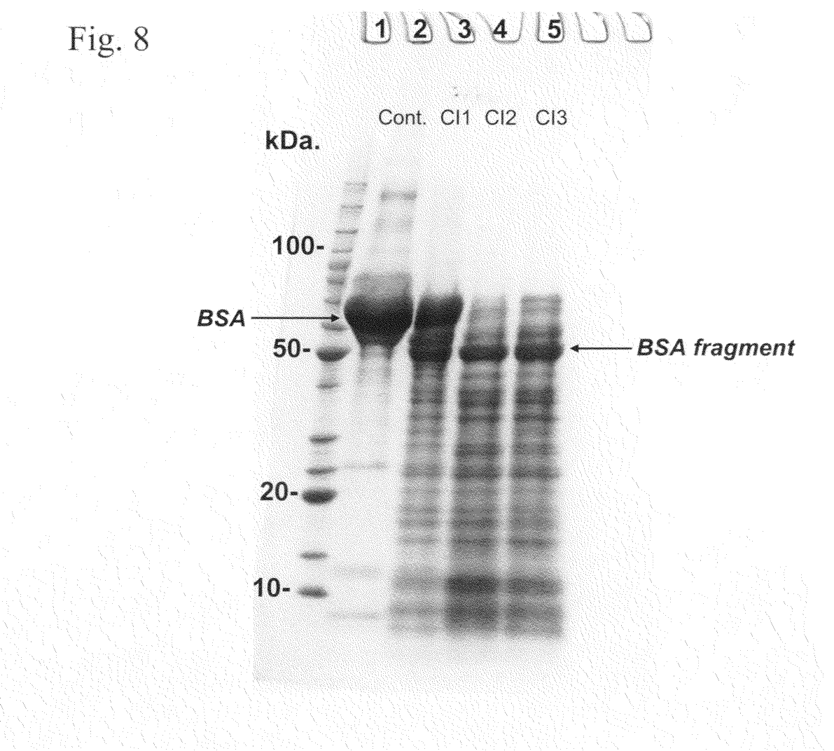

Protein production by the infected cells was confirmed by SDS-PAGE analysis and activity assays of whole-cell lysates or culture supernatants (Sambrook et al., Molecular Cloning A Laboratory Manual, Cold Spring Harbor, 1989). FIG. 8 shows SDS-Page analysis of conditioned media from virus infected Sf21 cells expressing a control protein or the chymopapain isoenzymes of the invention. Lane 2 contains media from control-virus infected cells. The control virus expressed β-galactosidase intracellularly. Lane 3 contains media from cells infected with recombinant baculovirus expressing the prepro form of CI1. Lane 4 contains media from cells infected with recombinant baculovirus expressing the prepro form of CI2. Lane 5 contains media from cells infected with recombinant baculovirus expressing the prepro form of CI3. The pro form of CI1, CI2, and CI3 was secreted to the media.

The culture media contained bovine fetal serum, which is rich in bovine serum albumin. The amount of chymopapain isoenzyme secreted to the media by the infected cells was too low to detect on the gel. The BSA present in the culture, however, was digested (FIG. 6, lanes 3, 4, and 5) indicating that the chymopapain isoenzymes were being secreted to the media and were active.

Protein Purification

Chymopapain isoenzymes comprising C-terminal histidine tags are purified from the supernatant or cell lysate by affinity chromatography using a metal chelating resin, such as, for example, a Ni-NTA column. Fractions are collected and analyzed by SDS-PAGE. Fractions containing protein are pooled and dialyzed overnight at 4° C. against 10 mM Tris-HCl (pH 8.0) and 200 mM NaCl (Mason et al., 2001, Protein Exp., 23:45-54). After dialysis the chymopapain isoenzymes are activated to obtain mature active protein.

Nontagged chymopapain isoenzymes are purified from the supernatant or cell lysate by anion exchange chromatography (U.S. Pat. No. 5,468,480; Mason et al., 2001, Protein Exp. Purif., 23: 45-54) or active site directed affinity chromatography using reversible chymopapain inhibitory peptides (U.S. Pat. No. 5,468,480). Fractions containing protein are pooled and dialyzed overnight at 4° C. against 10 mM Tris-HCl (pH 8.0) and 200 mM NaCl (U.S. Pat. No. 5,468,480; Mason et al., 2001, Protein Exp., 23:45-54). After dialysis the chymopapain isoenzymes are activated to obtain mature active protein.

### Example 3

### Expression of Pro Chymopapain Isoenzymes in E. coli

Competent cells, E. coli strain BL21 [DE3], were transformed with pET-28A expression vectors containing cDNA encoding the pro-form of CI3 (SEQ ID NO:3) as described in Example 1. Transformed BL21 [DE3] cells were grown at 37° C. with shaking (250 rpm) in baffled 2 liter flasks containing 500 ml of 2×YT culture media and 10 μg/ml kanamycin. Each flask was inoculated 1:50 (v/v) from an overnight culture produced at 37° C. When the culture OD₆₀₀ₙₘ reached ˜0.7, protein expression was induced with IPTG at a final concentration of 1 mM, and grown for an additional 3 hours at 37° C. The cells were harvested by centrifugation in a Sorvall GS3 rotor at 7000 rpm for 10 minutes at 4° C.

Protein production by the transformed BL21-[DE3] cells was confirmed by SDS-PAGE analysis. The cells were analyzed by SDS-PAGE for production of recombinant CI3 before and after IPTG induction. Cell pellets from 0.5 ml of culture were resuspended in 100 μl of reducing 2× Laemmli sample buffer [2× solution: 125 mM Tris(HCl) pH 6.8, 20% glycerol (v/v), 4% SDS (w/v), 5% β-ME (v/v), and 0.005% bromophenol blue (w/v)], and heated for 15-20 minutes at 90-100° C. with frequent vortexing. Proteins were separated on 4-20% polyacrylamide gradient mini-gels (Invitrogen Corp., Carlsbad, Calif., catalog #EC60255; 1 mm thick; 15 wells/gel) in 25 mM Tris base, 192 mM glycine, and 0.1% SDS (w/v) with 135 V constant voltage and the gels were stained with Coomassie brilliant blue.

Expression of the pro-form of CI3 (SEQ ID NO:3) was detected on the stained gel (data not shown). Cells transformed with expression vectors coding for CI3 produced an ˜38 kDa. polypeptide after IPTG induction as compared to a control, demonstrating the transformed cells were producing the pro-form of CI3 (data not shown). Solubility analysis indicated that the recombinant CI3 was denatured and likely sequestered in cytoplasmic inclusion bodies (data not shown).

### Example 4

Analysis of Novel Chymopapain Isoenzymes for Cysteine Protease Activity

Characterization of the pro-CI3 produced by E. coli indicated the pro-protein was denatured and sequestered in cytoplasmic inclusion bodies. Therefore, the pro-protein was purified from the inclusion bodies, refolded, and activated to generate the mature protein using established methods [See Taylor, et al., Plant Science 145 (1999) 41-47 and references therein].

Purification

Recombinant pro-CI3 was recovered from the E. coli inclusion bodies by centrifugation. The pro-CI3 was produced in E. coli as described in Example 3. Pellets from 2.4 liters of cell culture were washed twice in 100 ml of 20 mM Tris(HCl) pH 8.05, 150 mM NaCl, and 5 mM EDTA (TBS-E) and collected by centrifugation at 13,000 rpm for 10 minutes at 4° C. After the second wash the cells were resuspended in 100 ml TBS-E with 20 mM β-ME and lysed by sequential addition of lysozyme at a final concentration of 0.2 mg/ml (w/v), 1 ml of 10% Triton X-100, benzonase (Novagen, cat. # 70664-3) at a final concentration of 8 U/ml, and 9 ml of 10% Triton X-100. After each addition, the mixture was briefly homogenized (Virtis, Inc., Gardiner, N.Y., Handishear) and incubated at room temperature for about 10 minutes. Inclusion body protein was sedimented by centrifugation at 16,000 rpm for 20 minutes at 4° C. The supernatants were removed and the pellets were washed once with 100 ml of TBS-E containing Triton X-100 at a final concentration of 1% (v/v) followed by two washes with 100 ml of ultrapure water containing 0.1% Triton X-100 (v/v). Each wash consisted of resuspending the pellets in the wash solution, homogenizing, and sedimenting the insoluble material by centrifugation at 16,000 rpm for 20 minutes at 4° C. The final pellets were resuspended in 25 ml of ultrapure water. The total protein in each supernatant and pellet was examined by SDS-PAGE analysis. Standard PAGE methods were used.

Refolding

The washed inclusion body protein (100-200 mg) was resuspended in 10 ml of 50 mM Tris-acetate pH 8.6, 6 M guanidine HCl, 1 mM EDTA, and 10 mM DTT and incubated at 37° C. for one hour. The solution was then diluted 1:10 with 0.5 M Tris-acetate pH 8.6, 6 M guanidine HCl, 1 mM EDTA, and 0.1 M oxidized glutathione and incubated overnight at 4° C. (final volume of 100 ml, 16-20 hours). In the morning, the solubilized protein was diluted 1:50 into refolding buffer (100 mM Tris-acetate pH 8.2 containing 0.4 M L-arginine and 3 mM L-cysteine) and incubated at room temperature for 2 hours (final volume of 2.5 liters). The dilute refolding solution was concentrated to about 75 ml and the refolding buffer was exchanged for activation buffer (100 mM acetate pH 5 containing 20 mM L-cysteine) using an ultrafiltration/diafiltration membrane cartridge (Amersham Biosciences, Piscataway, N.J., model # UFP-10-C-4MA) at 4° C. The concentrated protein solution was clarified by centrifugation at 16,000 rpm for 20 minutes at 4° C. Refolded pro-chymopapain in the supernatant was examined by SDS-PAGE (see below).

Activation and Purification of Mature Protein

Refolded recombinant pro-CI3 was activated by adjusting the pH of the solution with acetic acid to pH 4.0 and incubating the solution in a 60° C. water bath for 30 minutes. Cleaved pro-sequence and any remaining pro-CI3 were separated from mature CI3 by cation exchange chromatography. The activated protein solution was cooled on ice and then loaded to a cation exchange column (Bio-Rad Laboratories, Inc., Hercules, Calif., Macro-Prep HighS Support, 5 ml cartridge, catalog #732-0066) at 4° C. The column was washed with 50 ml of 50 mM sodium phosphate pH 6.0, 20 ml of 50 mM sodium phosphate pH 6.0 containing 100 mM NaCl, 6 ml of 50 mM sodium phosphate pH 6.0 containing 200 mM NaCl and mature CI3 was eluted with 45 ml of 50 mM sodium phosphate pH 6.0 containing 400 mM NaCl. The fractions were analyzed by SDS-PAGE.

SDS-PAGE Analysis

Samples containing refolded pro-CI3 or mature CI3 required special handling to prevent proteolysis of all proteins present in the aliquot for SDS-PAGE, including refolded pro-CI3 and mature CI3. E64 (Sigma-Aldrich, St. Louis, Mo., catalog #E3132) was added to 50 μl of protein solution to a final concentration of 280 μM and incubated at room temperature for 5 minutes. The total protein in the sample was precipitated with 50 μl of 10% trichloroacetic acid (v/v) on ice for 10 minutes and collected by centrifugation at 13,000 rpm for 10 minutes at 4° C. The protein in the pellets was resuspended in 10 μl of 2× Laemmli sample buffer and 2 μl of 1 M Tris. The samples were not heated. The proteins were separated on 4-20% polyacrylamide gradient mini-gels (Invitrogen Corp., Carlsbad, Calif., catalog #EC60255; 1 mm thick; 15 wells/gel) in 25 mM Tris base, 192 mM glycine, and 0.1% SDS (w/v) with 135 V constant voltage. The gels were stained with Coomassie brilliant blue and analyzed to determine the purity of mature CI3 in the fractions.

After SDS-PAGE analysis, the fractions containing the highest purity mature CI3 were pooled. The volume of the pooled protein was decreased using a centrifugal device with a 9,000 Dalton molecular weight cut off (iCON concentrator, Pierce Chemical Company, Rockford, Ill., catalog #89885). When the volume was reduced to about 2.5 ml, 12 ml of 20 mM sodium phosphate pH 7.2, 150 mM NaCl with 10 mM L-cysteine was added and the volume of the concentrate was reduced to about 1.5 ml. Buffer exchange was accomplished by repeating this step twice. The concentration of mature CI3 was determined by A280 nm using an extinction coefficient of 1.83 ml mg⁻¹ cm⁻¹.

Cysteine Protease Activity Assay

The cysteine protease activity of mature CI3 was measured in flat-bottomed 96-well plates using N_{α}-Benzoyl-D,L-arginine 4-nitroanilide hydrochloride (BAPNA, Sigma-Aldrich, St. Louis, Mo., catalog #B4875) as the substrate. Release of p-nitroaniline, a traditional measure of the enzymatic activity of chymopapain, was determined by absorption spectrometry.

The assay was performed essentially as described in U.S. Pat. No. 5,468,480 (assay method no. 1, Smith Assay) with volumes scaled for use in a 96-well plate. Purified chymopapain from papaya latex (Sigma-Aldrich, St. Louis, Mo., catalog #C8526) was used as a positive control. Briefly, 4 μg of mature CI3 or purified chymopapain from papaya latex (positive control) was added to 50 mM sodium acetate pH 6.0, 2.5 mM L-cysteine, and 1 mM EDTA in a 96-well plate, mixed, and incubated at 37° C. for 5 minutes. BAPNA was added at a final concentration of 1 mM, mixed, and incubated at 37° C. At 30 minutes after substrate addition, the absorbance at 405 nm was measured with a plate reader (Bio-Tek Instruments, Inc., Winooski, Vt., Model #EL312E) to determine the amount of p-nitroaniline released. Cysteine protease activity was calculated from the absorbance data as described in U.S. Pat. No. 5,468,480 (assay method no. 1, Smith Assay).

Mature CI3 generated from three separate lots of recombinant CI3 was tested. CI3 from all three lots had cysteine protease activity (Table 4). The specific activity of the recombinant CI3 was approximately 10 fold greater than the specific activity of the chymopapain purified from papaya latex (Table 4).

TABLE 4

| | Amount | | Specific Activity² |
|---|---|---|---|
| | Tested (μg) | A_{405 nm}¹ | (pmol/s/mg) |
| Negative | | 0.110 | |
| Control | | | |
| CI3, Lot No. 1 | 4 | 0.436 | 1027.5 |
| CI3, Lot No. 2 | 4 | 0.388 | 877.5 |
| CI3, Lot No. 3 | 4 | 0.432 | 1016.4 |
| Purified | 4 | 0.141 | 97.9 |
| Chymopapain | 16 | 0.2 | 71.0 |
| (papaya latex) | | | |

¹Average absorbance at 405 nm, n = 2.
²Amount of p-nitro aniline formed per second per mg of protein.

### Deposit of Material

The following materials were deposited with the American Type Culture Collection (ATCC), 10801 University Blvd., Manassas, Va. 20110-2209, USA:

| Material | ATCC Deposit No. | Deposit Date |
|---|---|---|
| pET28-ST5 | PTA-6144 | Aug. 4, 2004 |
| pET28-ST6 | PTA-6145 | Aug. 4, 2004 |

The deposit was made under the provisions of the Budapest Treaty on the International Recognition of the Deposit of Microorganisms for the Purpose of Patent Procedure and the Regulations thereunder. Plasmid pET28-ST5 contains a nucleic acid sequence that encodes CI3 (SEQ ID NO:5). Plasmid pET28-ST6 contains a nucleic acid sequence that encodes CI2 (SEQ ID NO:3).

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

1-57. (canceled)

58. A method for treating a disc disorder, comprising locally administering recombinant chymopapain and/or synthetic chymopapain to a disc.

59. A method according to claim 58, wherein the disc disorder comprises increased intradiscal pressure, herniated nucleus pulposus, extruded nucleus pulposus, degenerative disc disease, nerve root compression or stretching, sciatica, or scoliosis.

60. A method according to claim 58, wherein intradiscal pressure is increased due to trauma to the spine, sciatica, herniated lumbar disc disease, or nerve root compression or stretching.

61. A method according to claim 58, wherein the herniated nucleus pulposus is associated with trauma to the spine, nerve root compression or stretching, sciatica, herniated lumbar disc disease, or chronic low back pain.

62. A method according to claim 58, wherein the disc is a thoracic, cervical, lumbar, or lumbosacral intervertebral disc.

63. A method for reducing intradiscal pressure in an intervertebral spinal disc, comprising locally administering recombinant chymopapain and/or synthetic chymopapain to the disc.

64. A method according to claim 63, wherein the disc is a thoracic, cervical, lumbar, or lumbosacral intervertebral disc.

65. A method according to claim 63, wherein the disc is herniated.

66. A method according to claim 63, wherein the intradiscal pressure is increased due to trauma to the spine, sciatica, herniated lumbar disc disease, or nerve root compression or stretching.

67. A method for treating a herniated intervertebral spinal disc, comprising locally administering recombinant chymopapain and/or synthetic chymopapain to the herniated disc.

68. A method according to claim 67, wherein the herniated disc is a lumbar or lumbosacral intervertebral disc.

69. A method according to claim 67, where the herniated disc is associated with trauma to the spine, sciatica, herniated lumbar disc disease, or chronic low back pain.

70. A method for removing a nucleus pulposus of an intervertebral spinal disc, comprising locally administering recombinant chymopapain and/or synthetic chymopapain to the nucleus pulposus.

71. A method according to claim 70, wherein the nucleus pulposus is removed to prepare a nuclear space in the disc for insertion of a nucleus pulposus replacement device.

72. A method for decreasing spinal stiffness, comprising locally administering recombinant chymopapain and/or synthetic chymopapain to a disc at the level of the spinal stiffness or curvature of the spine.

73. A method according to claim 72, wherein the spinal stiffness or curvature of the spine is associated with scoliosis.

74. A method according to claim 58, wherein locally administering recombinant chymopapain and/or synthetic chymopapain to a disc comprises contacting a nucleus pulposus of the disc with the recombinant or synthetic chymopapain.

75. A method according to claim 74, wherein contacting comprises:
(a) inserting a delivery device into a nucleus pulposus of the disc;
(b) confirming placement of the delivery device with an imaging device; and
(c) delivering the recombinant or synthetic chymopapain into the nucleus pulposus.

76. A method according to claim 75, wherein the delivery device is inserted into the nucleus pulposus percutaneously or into the nucleus pulposus through a surgical incision.

77. A method according to claim 75, wherein the delivery device is a canula, endoscope with operating channel, catheter, or needle.

78. A method according to claim 75, wherein the imaging device is x-ray, ultrasound, computerized topography scanner, or magnetic resonance imager.

79. A method according to claim 58, wherein the nucleus pulposus is dissolved.

80. A method according to claim 58, wherein the recombinant chymopapain and/or synthetic chymopapain is in admixture with a pharmaceutically acceptable carrier.

81. A method according to claim 58, wherein the recombinant chymopapain and/or synthetic chymopapain is in unit dosage form.

82. A method according to claim 58, wherein the recombinant chymopapain or synthetic chymopapain comprises an amino acid sequence that has at least about 80% amino acid sequence identity with SEQ ID NO:16 or SEQ ID NO:17 and cysteine protease activity.

83. A method according to claim 82, wherein the recombinant chymopapain or synthetic chymopapain comprises:
a) one or more amino acid substitutions at residues corresponding to amino acid residues 1, 130, 167, 168, 170, 186, 197, 202, 207, 217, 241, 292, 293, or 309 of SEQ ID NO:16, or
b) one or more amino acid substitutions at residues corresponding to amino acid residues 21, 58, 59, 61, 77, 88, 93, 98, 108, 132, 183, 184, or 200 of SEQ ID NO:17.

84. An method according to claim 83, wherein the substitution comprises:
a) one or more of A21S, K58R, H₅₉N, Y61H, N77D, Y88C, Y93C, T98E, T108S, V132F, P183N, N184K, and S200C, or
b) one or more of A1M, A130S, K167R, H168N, Y170H, N186D, Y197C Y202C, T207E, T217S, V241F, P292S, N293K, and S309C.

85. A method according to claim 82, wherein the recombinant chymopapain or synthetic chymopapain comprises an amino acid sequence of SEQ ID NO:10, SEQ ID NO:11, SEQ ID NO:19, SEQ ID NO:21, SEQ ID NO:23, or SEQ ID NO:24.

86. A method according to claim 82, wherein the recombinant chymopapain or synthetic chymopapain comprises an amino acid sequence of SEQ ID NO:25.

87. A method according to claim 82, wherein the recombinant chymopapain or synthetic chymopapain comprises an amino acid sequence of SEQ ID NO:1, SEQ ID NO:3, SEQ ID NO:5, SEQ ID NO: 8, SEQ ID NO:9, SEQ ID NO:10, or SEQ ID NO:11.

88. An isolated chymopapain isoenzyme, comprising:
a) substitution of one or more amino acids corresponding to residues 1, 130, 168, 170, 186, 202, 207, 217, 292, 293, or 309 of SEQ ID NO:16, wherein the isoenzyme has at least 80% amino acid sequence identity with SEQ ID NO: 16 and cysteine protease activity, or
b) substitution of one or more amino acids corresponding to residues 21, 59, 61, 77, 93, 98, 108, 183, 184, or 200 of SEQ ID NO:17, wherein the isoenzyme has at least 80% amino acid sequence identity with SEQ ID NO: 17 and cysteine protease activity.

89. A chymopapain isoenzyme according to claim 88, wherein the substitution comprises:
a) one or more of A1M, A130S, H168N, Y170H, N186D, Y202C, T207E, T217S, P292S, N293K, or S309C, or
b) one or more of A21S, H₅₉N, Y61H, N77D, Y93C, T98E, T108S, P183N, N184K, and S200C.

90. A chymopapain isoenzyme according to claim 88, wherein the isoenzyme comprises an amino acid sequence of SEQ ID NO:1, SEQ ID NO:3, SEQ ID NO:5, SEQ ID NO:24, or SEQ ID NO:25.

91. A chymopapain isoenzyme according to claim 88, wherein the isoenzyme comprises cysteine protease activity that is greater than the cysteine protease activity of refined chymopapain in a 1 mM N_{α}-Benzoyl-D,L-arginine 4-nitroanilide hydrochloride (BAPNA) assay at 37° C., pH 6.0.

92. A chymopapain isoenzyme according to claim 91, wherein the cysteine protease activity of the isoenzyme is at least 10 fold greater than the cysteine protease activity of the refined chymopapain.

93. A polynucleotide encoding a chymopapain isoenzyme according to claim 88.

94. A polynucleotide according to claim 93, wherein the polynucleotide comprises a nucleic acid sequence of SEQ ID NO:2, SEQ ID NO:4, or SEQ ID NO:6.

95. A vector comprising a polynucleotide according to claim 93.

96. A host cell comprising a vector according to claim 93.

97. A host cell according to claim 96, wherein the host cell is a yeast cell, an insect cell, or a mammalian cell.

98. A composition, comprising a chymopapain isoenzyme according to claim 88 in admixture with a physiologically acceptable carrier.

99. An antibody that specifically binds a chymopapain isoenzyme according to claim 88.

100. A method of treating a degenerative disc disease, comprising administering recombinant or synthetic chymopapain to a diseased disc to evacuate nucleus pulposus from the nuclear space in the disc and inserting a nuclear replacement material into the evacuated nuclear space.
